SEQUENCE LISTING (7 sequences)

SEQ ID NO: 1 - DNA, 57 bp, synthetic construct
  source 1..57: mol_type = other DNA
  variation 57: note = N is A, T, C, or G
  variation 56: note = N is A, C, or G
aagcagtggtatcaacgcagagtacttttttttttttttttttttttttttttttvn

SEQ ID NO: 2 - RNA, 14 nt, synthetic construct
  source 1..14: mol_type = other RNA
aaaaaaaaaaaaaa

SEQ ID NO: 3 - RNA, 22 nt, synthetic construct
  source 1..22: mol_type = other RNA
  misc_feature 18..19: note = adapter
aaaaaaaaaaaaaaccccgggg

SEQ ID NO: 4 - RNA, 19 nt, synthetic construct
  source 1..19: mol_type = other RNA
aaaaaaaaaaaaaaaaccc

SEQ ID NO: 5 - RNA, 21 nt, synthetic construct
  source 1..21: mol_type = other RNA
aaaaaaaaaaaaaaaaaaaaa

SEQ ID NO: 6 - RNA, 58 nt, synthetic construct
  source 1..58: mol_type = other RNA
  misc_feature 1: note = Biotin Tag = /5Biosg/
  misc_feature 58: note = 3SpC3
aagcagtggtatcaacgcagagtcagaatttttttttttttttttttttttttttttt

SEQ ID NO: 7 - RNA, 59 nt, synthetic construct
  source 1..59: mol_type = other RNA
  misc_feature 1: note = Biotin Tag = /5Biosg/
  misc_feature 59: note = 3SpC3
aagcagtggtatcaacgcagagrtcagaatttttttttttttttttttttttttttttt

(19) United States
(12) Patent Application Publication
(10) Pub. No.: US 2025/0011859 A1
(43) Pub. Date: Jan. 9, 2025

(54) COMPOSITIONS AND METHODS FOR END TO END CAPTURE OF MESSENGER RNAS

(71) Applicants: THE BROAD INSTITUTE, INC., Cambridge, MA (US); THE GENERAL HOSPITAL CORPORATION, Boston, MA (US)

(72) Inventors: Zachary ZWIRKO, Cambridge, MA (US); Nir HACOHEN, Boston, MA (US); Aziz AL’KHAFAJI, Cambridge, MA (US)

(73) Assignees: THE BROAD INSTITUTE, INC., Cambridge, MA (US); THE GENERAL HOSPITAL CORPORATION, Boston, MA (US)

(21) Appl. No.: 18/750,283

(22) Filed: Jun. 21, 2024

Related U.S. Application Data
(63) Continuation of application No. PCT/US2022/082267, filed on Dec. 22, 2022.
(60) Provisional application No. 63/292,737, filed on Dec. 22, 2021.

(51) Int. Cl. C12Q 1/6869

(52) U.S. Cl. CPC C12Q 1/6869

(57) ABSTRACT

Methods and compositions for a single- or multi-pot protocol for the efficient end to end capture of RNAs (inclusive of their poly-A tail or their 3′ end) is described. Capture oligonucleotides containing a 3′ non-extendable end and a selectively cleavable base upstream of an oligo-dT or oligo-dN and a 5′ sequence containing unique molecular identifiers, and 2) a deoxyuracil glycosylase that acts only on a deoxyuracil present in a DNA: DNA duplex or DNA/RNA heteroduplex are used. A dual template switching mechanism may be used.

### CROSS-REFERENCE TO RELATED APPLICATIONS

The present application is a Continuation of International Application No. PCT/US2022/082267, filed Dec. 22, 2022 and claims the benefit of U.S. Provisional Application No. 63/292,737, filed Dec. 22, 2021. The entire contents of the above-identified application are hereby fully incorporated herein by reference.

### SEQUENCE LISTING

This application contains a sequence listing in electronic form as an xml file entitled BROD-5470WP_ST26.xml with size 9,350 bytes created on Dec. 21, 2022. The content of the sequence listing is incorporated herein in its entirety.

### TECHNICAL FIELD

The subject matter disclosed herein is generally directed to a protocol for the efficient end to end capture of mRNAs (inclusive of their poly-A tail) that can be performed in a single-pot reaction or using separate reactions.

### BACKGROUND

The transcriptome has been extensively studied in the age of next-generation sequencing (NGS), with the exception of the detailed composition of poly(A) tails because the current NGS platforms cannot handle homopolymeric sequences longer than 30 nucleotides (nt) by using a standard base-calling algorithm (Liu Y., Nie H., Liu H., Lu F.) Poly(A) inclusive RNA isoform sequencing (PAIso-seq) reveals wide-spread non-adenosine residues within RNA poly(A) tails. Nat Commun. 2019; 10 (1): 5292). Smart-seq2, one of the most sensitive single-cell RNA-sequencing (RNA-seq) technology, uses 3′-untranslated region (UTR) anchored oligo-dT primer (5′-AAGCAGTGGTATCAACGCAGAGTACT30VN-3′ (SEQ ID NO: 1), where “N” is A, T, C, or G and “V” is A, C, or G) for reverse transcription to construct the complementary DNA (cDNA) library. Id. The two terminal nucleotides “N” and “V” anchor the reverse transcriptase (RT) primer to the end of 3′-UTR and discard the poly(A) tails from the final cDNA library to avoid the homopolymeric sequences (Picelli, S. et al. Full-length RNA-seq from single cells using Smart-seq2. Nat. Protoc. 9, 171-181 (2014)). Other commonly used RNA-seq tools also ignore or discard poly(A) sequences during library preparation, sequencing, or data analysis steps.

Prior methods exist to capture the full-length mRNAs (FLAM-seq, PAISO-seq) however these methods are multi-step protocols, not amenable to streamlined reactions such as droplet based single-cell RNA sequencing (Liu Y, Nie H, Liu H, Lu F., Poly(A) inclusive RNA isoform sequencing (PAIso-seq) reveals wide-spread non-adenosine residues within RNA poly(A) tails. Nat Commun. 2019; 10 (1): 5292; and Legnini I, Alles J, Karaiskos N, Ayoub S, Rajewsky N. FLAM-seq: full-length mRNA sequencing reveals principles of poly(A) tail length control. Nat Methods. 2019; 16 (9): 879-886). Thus, there is a need for a single-pot protocol for the efficient end to end capture of mRNAs (inclusive of their poly-A tail).

Citation or identification of any document in this application is not an admission that such a document is available as prior art to the present invention.

### SUMMARY

In one aspect, the present disclosure provides for a system for capturing full-length RNAs as cDNA, said system comprising: a single stranded capture oligonucleotide comprising from 3′ to 5′: 1) a non-extendable end, 2) a capture sequence, 3) a sequence comprising a selectively cleavable base that can be cleaved in a DNA: DNA duplex or DNA/RNA heteroduplex, 4) a sequence comprising one or more barcode sequences, and 5) a terminal adapter sequence; an enzyme or combination of enzymes capable of cleaving the selectively cleavable base only in a DNA: DNA duplex or DNA/RNA heteroduplex; deoxyribonucleotide triphosphates (dNTPs); a reverse transcriptase (RT); and a plurality of RNAs. In certain embodiments, the sequence comprises a selectively cleavable base is a dU sequence. In certain embodiments, the enzyme or combination of enzymes is a deoxyuracil glycosylase that only has activity on a deoxyuracil present in a DNA: DNA duplex or DNA/RNA heteroduplex and an endonuclease capable of cleavage of an abasic site. In certain embodiments, the deoxyuracil glycosylase is a family 5 UDGb. In certain embodiments, the family 5 UDGb comprises an AllIN mutation in the same position as in the family 5 UDGb from Thermus thermophiles. In certain embodiments, the endonuclease is endonuclease VIII. In certain embodiments, the endonuclease is endonuclease IV. In certain embodiments, the endonuclease IV is Thermus thermophilus (Tth) endonuclease IV. In certain embodiments, the sequence comprising a selectively cleavable base is a ribobase comprising sequence. In certain embodiments, the enzyme or combination of enzymes is RNAseH2. In certain embodiments, the capture sequence is an oligo-dT sequence and the plurality of RNAs are a plurality of mRNAs. In certain embodiments, the capture sequence is an oligo-dN sequence and the plurality of RNAs are a plurality of non-polyadenylated RNAs. In certain embodiments, the oligo-dN sequence is specific for a non-polyadenylated RNA, optionally, a lncRNA, miRNA, or rRNA. In certain embodiments, the oligo-dN sequence is a degenerate/random sequence.

In certain embodiments, the system is comprised in an aqueous discrete volume. In certain embodiments, the system is comprised in more than one aqueous discrete volume, wherein a first aqueous discrete volume comprises at least a single stranded capture oligonucleotide a plurality of RNAs, optionally, a single stranded capture oligonucleotide and dNTPs, an RT, and RNAs, and subsequent aqueous discrete volumes comprise one or more of an enzyme or combination of enzymes capable of cleaving the selectively cleavable base, dNTPs, and an RT, and any intermediate reaction product. In certain embodiments, the aqueous discrete volume or first aqueous discrete volume comprises a plurality of capture oligonucleotides, wherein the one or more barcode sequences for each capture oligonucleotide is a Unique Molecular Identifier (UMI) that is different for each capture oligonucleotide in the plurality of capture oligonucleotides.

In another aspect, the present disclosure provides for a system for capturing full-length RNAs as cDNA, wherein the system comprises a plurality of aqueous discrete volumes or first aqueous discrete volumes according to any embodiment herein, wherein the one or more barcodes for each capture oligonucleotide further comprises a cell barcode that is the same among capture oligonucleotides in an aqueous discrete volume, but is different among capture oligonucleotides in any other aqueous discrete volume. In certain embodiments, the aqueous discrete volume is a microwell or a droplet.

In certain embodiments, the capture oligonucleotide or plurality of capture oligonucleotides is attached to a solid support through a linker attached at the 5′ end of the capture oligonucleotides. In certain embodiments, the linker is cleavable. In certain embodiments, the solid support is a bead. In certain embodiments, each aqueous discrete volume comprises no more than one bead. In certain embodiments, the solid support is a slide and each capture oligonucleotide comprises a spatial barcode that identifies the location of the capture oligonucleotide on the slide.

In certain embodiments, the system further comprises a template switching oligo (TSO) comprising an adapter sequence. In certain embodiments, the TSO comprises a locked nucleic acid (LNA). In certain embodiments, the TSO comprises a 3′-deoxyguanosine.

In another aspect, the present disclosure provides for a system for capturing full-length RNAs as cDNA, said system comprising an aqueous discrete volume comprising: a single stranded capture oligonucleotide capable of priming extension of RNA, said capture oligonucleotide comprising from 3′ to 5′: 1) a non-extendable end, and 2) a capture sequence; a template switching oligo (TSO) capable of being extended at its 3′ end, said TSO comprising from 3′ to 5′: 1) a sequence comprising 3 guanosine bases, 2) a sequence comprising one or more barcode sequences, and 3) a terminal adapter sequence; deoxyribonucleotide triphosphates (dNTPs); a reverse transcriptase; and a plurality of RNAs. In certain embodiments, the capture sequence is an oligo-dT sequence and the plurality of RNAs are a plurality of mRNAs. In certain embodiments, the capture sequence is an oligo-dN sequence and the plurality of RNAs are a plurality of non-polyadenylated RNAs. In certain embodiments, the oligo-dN sequence is specific for a non-polyadenylated RNA, optionally, a lncRNA, miRNA, or rRNA. In certain embodiments, the oligo-dN sequence is a degenerate/random sequence. In certain embodiments, the aqueous discrete volume comprises a plurality of TSOs, wherein the one or more barcode sequences for each TSO is a Unique Molecular Identifier (UMI) that is different for each TSO in the plurality of TSOs.

In another aspect, the present disclosure provides for a system for capturing full-length RNAs as cDNA, wherein the system comprises a plurality of aqueous discrete volumes according to any embodiment herein, wherein the one or more barcodes for each TSO further comprises a cell barcode that is the same among TSOs in an aqueous discrete volume, but is different among TSOs in any other aqueous discrete volume. In certain embodiments, the aqueous discrete volume is a microwell or a droplet. In certain embodiments, the plurality of TSOs is attached to a solid support through a linker attached at the 5′ end of the TSO. In certain embodiments, the linker is cleavable. In certain embodiments, the solid support is a bead. In certain embodiments, each aqueous discrete volume comprises no more than one bead. In certain embodiments, the solid support is a slide and the TSO comprises a spatial barcode that identifies the location of the TSO on the slide.

In another aspect, the present disclosure provides for a method of capturing full-length RNAs comprising incubating an aqueous discrete volume or one or more of the more than one aqueous discrete volumes according to any embodiment herein at one or more temperatures such that mRNA is extended into the capture oligonucleotide by reverse transcriptase, the selectively cleavable base is cleaved in the extended double strand sequence, and the cleaved capture oligonucleotide is extended by reverse transcriptase using the RNA as a template, wherein the method takes place in a single aqueous discrete volume; or wherein the method takes place in more than one aqueous discrete volume with or without intervening purification, whereby full-length RNAs are captured as cDNA in a single reaction or multiple independent reactions. In certain embodiments, the method further comprises: contacting the cDNA with a terminal deoxynucleotidyl transferase (TdT), poly(A) polymerase, or poly(U) polymerase to add nucleotides to the 3′ end of the cDNA to obtain tailed cDNA; and contacting the tailed cDNA with an adapter sequence comprising an overhang complementary to the nucleotides added in (a) and a ligase, whereby full-length RNAs are captured as cDNA comprising adapters at both ends. In certain embodiments, the adapter is a hairpin adapter.

In another aspect, the present disclosure provides for a method of capturing full-length RNAs comprising incubating an aqueous discrete volume or one or more of the more than one aqueous discrete volumes according to any embodiment herein at one or more temperatures such that RNA is extended into the capture oligonucleotide by reverse transcriptase, the selectively cleavable base is cleaved in the extended double strand sequence, the capture oligonucleotide is extended by reverse transcriptase using the RNA as a template, and template switching occurs after the RNA is reverse transcribed, wherein the method takes place in a single aqueous discrete volume; or wherein the method takes place in more than one aqueous discrete volume with or without intervening purification, whereby full-length RNAs are captured as cDNA in a single reaction or multiple independent reactions.

In another aspect, the present disclosure provides for a method of capturing full-length RNAs comprising incubating an aqueous discrete volume according to any embodiment herein at one or more temperatures such that the template switching oligo performs template switching activity from an RNA extension product templated from the non-extendable capture oligonucleotide, followed by extension from the template switch oligo templating from the RNA, synthesizing full length cDNA, whereby full-length RNAs are captured as cDNA in a single reaction.

In another aspect, the present disclosure provides for a plurality of beads comprising single stranded capture oligonucleotides attached to the beads at the 5′ end comprising from 3′ to 5′: 1) a non-extendable end, 2) a capture sequence, 3) a sequence comprising a selectively cleavable base that can be cleaved in a DNA: DNA duplex or DNA/RNA heteroduplex, 4) a sequence containing one or more barcode sequences, and 5) a terminal adapter sequence. In certain embodiments, the one or more barcode sequences for each capture oligonucleotide is a Unique Molecular Identifier (UMI) that is different for each capture oligonucleotide on any one bead. In certain embodiments, the one or more barcodes for each capture oligonucleotide further comprises a cell barcode that is the same among capture oligonucleotides on any one bead, but is different among capture oligonucleotides on any other bead. In certain embodiments, the single stranded capture oligonucleotides are attached to the beads through a linker attached at the 5′ end of the single stranded capture oligonucleotides. In certain embodiments, the linker is cleavable. In certain embodiments, the sequence comprising a selectively cleavable base is a dU sequence. In certain embodiments, the sequence comprising a selectively cleavable base is a ribobase comprising sequence.

In another aspect, the present disclosure provides for a plurality of beads comprising template switching oligos (TSOs) attached to the beads at the 5′ end and capable of being extended at its 3′ end, said TSOs comprising from 3′ to 5′: 1) a sequence comprising 3 guanosine bases, 2) a sequence comprising one or more barcode sequences, and 3) a terminal adapter sequence. In certain embodiments, the one or more barcode sequences for each TSO is a Unique Molecular Identifier (UMI) that is different for each TSO on any one bead. In certain embodiments, the one or more barcodes for each TSO further comprises a cell barcode that is the same among capture oligonucleotides on any one bead, but is different among TSOs on any other bead. In certain embodiments, the TSOs are attached to the beads through a linker attached at the 5′ end of the TSOs. In certain embodiments, the linker is cleavable.

In another aspect, the present disclosure provides for a slide comprising single stranded capture oligonucleotides attached to the slide at the 5′ end comprising from 3′ to 5′: 1) a non-extendable end, 2) a capture sequence, 3) a sequence comprising a selectively cleavable base that can be cleaved in a DNA: DNA duplex or DNA/RNA heteroduplex, 4) a sequence containing one or more barcode sequences, and 5) a terminal adapter sequence. In certain embodiments, the one or more barcode sequences for each capture oligonucleotide is a Unique Molecular Identifier (UMI) that is different for each capture oligonucleotide on the slide. In certain embodiments, the one or more barcodes for each capture oligonucleotide further comprises a spatial barcode that identifies the location of the capture oligonucleotide on the slide. In certain embodiments, the single stranded capture oligonucleotides are attached to the slide through a linker attached at the 5′ end of the single stranded capture oligonucleotides. In certain embodiments, the linker is cleavable. In certain embodiments, the sequence comprising a selectively cleavable base is a dU sequence. In certain embodiments, the sequence comprising a selectively cleavable base is a ribobase comprising sequence.

In another aspect, the present disclosure provides for a kit comprising the single stranded capture oligonucleotide or plurality of single stranded capture oligonucleotides of any embodiment herein or the plurality of beads of any embodiment herein or the slide of any embodiment herein. In certain embodiments, the kit further comprises a deoxyuracil glycosylase that only has activity on a deoxyuracil present in a DNA: DNA duplex or DNA/RNA heteroduplex. In certain embodiments, the deoxyuracil glycosylase is a family 5 UDGb. In certain embodiments, the family 5 UDGb comprises an AllIN mutation in the same position as in the family 5 UDGb from Thermus thermophiles. In certain embodiments, the kit further comprises endonuclease VIII or endonuclease IV. In certain embodiments, the kit further comprises RNAscH2.

In another aspect, the present disclosure provides for a kit comprising the single stranded capture oligonucleotide or plurality of single stranded capture oligonucleotides and TSOs of any embodiment herein or the plurality of beads of any embodiment herein.

In another aspect, the present invention provides for a template switching oligo (TSO) comprising a 3′-deoxyguanosine (3drG). In certain embodiments, the 3′ end of the TSO comprises a ribonucleotide, riboguanosine, and 3′-deoxyguanosine (rNrG3drG). In certain embodiments, the 3′ end of the TSO comprises two riboguanosines, and 3′-deoxyguanosine (rGrG3drG). In certain embodiments, the TSO further comprises a sequencing adaptor.

In another aspect, the present disclosure provides for a template switching system comprising: a template switching oligo according to any embodiment herein; a primer for first strand synthesis of a target RNA; a reverse transcriptase; and dNTP's. In certain embodiments, the primer comprises a poly-dT sequence.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—Schematic for mRNA end to end sequencing (mEE-seq) using an oligo-dT template and a template switching oligo (TSO) (SEQ ID NO: 2).

FIG. 2—Schematic for mRNA end to end sequencing (mEE-seq) using an oligo-dT template that includes an RNA polymerase promoter for amplification of full-length mRNA and a template switching oligo (TSO) (SEQ ID NO: 2).

FIG. 3—Schematic for mRNA end to end sequencing (mEE-seq) where the cDNA is 3′ end tailed and a hairpin adapter is ligated to the cDNA (SEQ ID NO: 2-3).

FIG. 4—Schematic for mRNA end to end sequencing (mEE-seq) using an oligo-dT template that includes an RNA polymerase promoter for amplification of full-length mRNA (SEQ ID NO: 2).

FIG. 5—Schematic for non-polyadenylated RNA end to end sequencing (mEE-seq) using a targeted capture/priming sequence.

FIG. 6—Schematic for non-polyadenylated RNA end to end sequencing (mEE-seq) using a random capture/priming sequence.

FIG. 7—Schematic for mRNA end to end sequencing (mEE-seq) using a dual TSO activity mechanism for full length mRNA capture (SEQ ID NO: 2, 4).

FIG. 8—RNAse H2 Titration Results. The addition of RNAse H2 significantly increases the amount of desired 452 base pair product.

FIG. 9A-9B—Ribonuclease Substrate Specificity. FIG. 9A. Product observed when a ribose base (RNA base) is replaced with a deoxy ribose base (DNA base) at the same position.

FIG. 9B. Expected cleavage events with ‘MEE-Seq’ primers containing either ribose or deoxyribose at the specified position. Primer sequences with 5′ and 3′ modifications shown below (SEQ ID NO: 5-7).

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes I X, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2^{nd} cd., J. Wiley & Sons (New York, N.Y. 1994), March, Advanced Organic Chemistry Reactions, Mechanisms and Structure 4^{th} ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present disclosure encompasses embodiments wherein the bodily fluid is selected from amniotic fluid, aqueous humour, vitreous humour, bile, blood serum, breast milk, cerebrospinal fluid, cerumen (carwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide compositions and methods for capturing full length mRNA molecules including the entire poly-A tail in a single reaction volume. In example embodiments, the compositions and methods can also be employed in multiple independent reactions with or without intervening purification. Prior to the present disclosure a single-pot protocol for the efficient end to end capture of mRNAs (inclusive of their poly-A tail) did not exist. Prior methods capture the full length mRNAs (FLAM-seq, PAISO-seq) using multi-step protocols, not amenable to streamlined reactions such as droplet based single-cell RNA sequencing or spatial capture technology. As described herein, mRNA end to end sequencing (mEE-seq), enables the efficient end to end capture of mRNAs from single-pot reactions, such as droplet based single-cell RNA sequencing. End to end mRNA sequencing is highly biologically informative as this provides both isoform level information, circumvents generation of artifactual truncated cDNAs formed via internal mRNA priming, as well as poly-A length which could serve as a temporal expression proxy. Using this read-out in the single cell format could enable a high resolution inference of RNA velocity.

The key innovations that allow the reaction to be performed in a single reaction include use of an RNA capture sequence to extend an RNA sequence past the end of the RNA sequence and to add additional sequence (e.g., barcodes, adapters), where generating double stranded DNA leads to the capture sequence being displaced from the RNA template, ensuring that during cDNA generation, the entire end of the RNA is captured.

In one example embodiment, the method includes: 1) use of an oligo-dT template containing a 3′ non-extendable end and an internal dU sequence upstream of the oligo-dT and a 5′ sequence containing unique molecular identifiers, cell barcodes (optional), and a terminal adapter sequence, 2) use of a deoxyuracil glycosylase that acts only on a deoxyuracil present in a DNA: DNA duplex or DNA/RNA heteroduplex, 3) priming and extension of mRNA on the template oligo described in point 1, 4) the excision of the dU base in the double extension product, leading to displacement extension from this newly formed 3′ end via a reverse transcriptase, and 5) reverse extension can continue till reaching the 5′ of the mRNA, where template switching can occur. Thus, because the deoxyuracil glycosylase acts only on double stranded DNA the oligo-dT template is not cleaved before being extended and the reactions can happen in a single reaction volume.

In one example embodiment, the method includes: 1) use of an oligo-dT template containing a 3′ non-extendable end and an internal ribobase sequence upstream of the oligo-dT and a 5′ sequence containing unique molecular identifiers, cell barcodes (optional), and a terminal adapter sequence, 2) use of a ribonuclease that selectively cleaves an RNA base in a DNA: DNA duplex, such as RNAseH2 or any other enzyme that will selectively cleave a ribose base in the context of a DNA: DNA duplex leaving a 3′ OH, 3) priming and extension of mRNA on the template oligo described in point 1, 4) the excision of the ribobase in the double extension product, leading to displacement extension from this newly formed 3′ end via a reverse transcriptase, and 5) reverse extension can continue till reaching the 5′ of the mRNA, where template switching can occur. Thus, because the ribonuclease acts only on double stranded DNA the oligo-dT template is not cleaved before being extended and the reactions can happen in a single reaction volume.

In one example embodiment, the method includes: 1) use of an oligo-dT template containing a 3′ non-extendable end, 2) use of a template switching oligo (TSO) containing 3 guanosine bases, a sequence comprising one or more barcode sequences, and a terminal adapter sequence, 3) priming and extension of mRNA on the template oligo described in point 1 via a reverse transcriptase, 4) template switching activity with the TSO and the RNA extension product templated from the blocked primer, 5) extension of the template switch oligo via a reverse transcriptase leading to displacement extension from this newly formed 3′ end, and 6) reverse extension can continue till reaching the 5′ of the mRNA, where template switching can occur. Thus, because the TSO can extend the mRNA after a template switching extension product is generated by extension of the oligo-dT template, the reactions can happen in a single reaction volume.

Systems for Capturing Full-Length mRNAs

In certain embodiments, the present disclosure provides for systems to capture full-length mRNA as cDNA. The systems can include a single aqueous volume where all steps in the process of using the systems can be performed, such that the systems do not require extraction steps, purification steps, or any steps to add additional reagents. The systems can also use the components of the systems to capture full-length mRNA as cDNA in separate reactions (e.g., aqueous volumes), such as 2 or 3 reactions, preferably, 2 reactions. For example, a first reaction can generate the RNA extension product using RNA, RT, and dNTP's and the second reaction can add the enzyme for cleavage of the capture oligonucleotide and extension by RT.

In one embodiment, a system uses a capture oligonucleotide having a base that can be selectively cleaved only when present in a double stranded sequence. In this example embodiment, the system relies on an end blocked RNA capture sequence that can be cleaved upstream of the end of the RNA sequence, such that extension of the entire RNA can then proceed.

In one embodiment, a system uses a dual template switching activity mechanism. In this example embodiment, the system relies on an end blocked RNA capture sequence that can bind to the 3′ end of a target RNA and template extension of the RNA by reverse transcriptase. The reverse transcriptase will add untemplated poly(C) nucleotides to the end of the extended RNA, which then allows binding of a template switching oligo (TSO) that includes one or more barcode sequences. The TSO can template extension of the RNA as well as prime extension using the RNA as a template. The TSO system is similar to the cleavage based system because in both systems the capture sequence is displaced upstream of the end of the RNA ensuring that the cDNA includes the entire full length RNA sequence. In the case of the TSO system, cleavage is not required because the capture sequence and TSO are already separate oligonucleotides.

### Aqueous Volumes

As used herein an “aqueous volume” refers to a water based volume where a biological/chemical/enzymatic reaction can occur. As used herein an aqueous volume can be a separate (i.e., discrete) aqueous volume present in a tube, well of a plate, microwell, microfluidic chamber, or droplet. An aqueous volume can also refer to the aqueous volume that allows reactions to take place on a surface, array or slide. A surface, array or slide may be partitioned to include more than one aqueous volume. Partitioning is meant to include actual physical separation and separation based only on the location of specific oligonucleotides on a surface, array or slide (e.g., each location of a surface, array or slide comprising a different spatial barcode can be referred to as a separate aqueous volume). In example embodiments, the system as described further herein can all be included in each of a plurality of aqueous volumes. As used herein, inactivation of a prior reaction in an aqueous volume and addition of new reagents to the aqueous volume can be referred to as a new aqueous volume.

### Capture Oligonucleotides

In example embodiments, the system includes single strand capture oligonucleotides that comprise capture sequences for target RNAs. In example embodiments, the capture oligonucleotides include a capture sequence for capturing full-length polyadenylated mRNAs. The capture sequence for capturing full-length polyadenylated mRNAs can include a poly-dT sequence (oligo-dT templates). In example embodiments, the capture oligonucleotides include a capture sequence for capturing non-polyadenylated RNAs, such as, but not limited to lncRNAs, miRNAs, and rRNAs. The capture sequence for capturing non-polyadenylated RNAs can include transcript specific sequences or a degenerate/random sequence (˜6-20 bp) (oligo-dN templates, where N can be any nucleotide sequence). In example embodiments, the system can include oligo-dN templates comprising different capture sequences specific for different non-polyadenylated RNAs (e.g., a mix of oligo-dN templates), such that multiple non-polyadenylated transcripts can be targeted simultaneously. As used herein, “oligo-dT template” or “oligo-dN template” can also be referred to as a “capture oligonucleotide” or a “primer” (i.e., oligo-dT primer, capture primer, oligo-dT dU primer, oligo-dN primer, oligo-dN dU primer). An oligo-dN template can be an oligo-dT template if the sequence includes a poly-dT sequence. In example embodiments, the oligo-dT templates include from 3′ to 5′: 1) a non-extendable 3′ end, 2) an oligo-dT sequence, 3) a sequence comprising a selectively cleavable base that can be cleaved in a DNA: DNA duplex or DNA/RNA heteroduplex (e.g., a deoxyuridine (dU) sequence or riboU sequence), 4) a sequence containing one or more barcode sequences, and 5) a terminal adapter sequence. In example embodiments, the oligo-dN templates include from 3′ to 5′: 1) a non-extendable 3′ end, 2) an oligo-dN sequence, 3) a sequence comprising a selectively cleavable base that can be cleaved in a DNA: DNA duplex or DNA/RNA heteroduplex (e.g., a deoxyuridine (dU) sequence or riboU sequence), 4) a sequence containing one or more barcode sequences, and 5) a terminal adapter sequence. In example embodiments, the capture oligonucleotides include from 3′ to 5′: 1) a non-extendable 3′ end, and 2) an oligo-dN sequence.

In example embodiments, the oligo-dT templates include a 3′ poly-dT sequence including about 30 dT nucleotides. In example embodiments, the oligo-dT template includes 5-10, 10-20, 20-30, 40-50 dT nucleotides. In example embodiments, the oligo-dN templates include a 3′ poly-dN sequence including about 30 dN nucleotides. In example embodiments, the oligo-dN template includes 5-10, 10-20, 20-30, 40-50 dN nucleotides. In preferred embodiments, the oligo-dN template includes about 6-20 nucleotides.

In example embodiments, the 3′ end is non-extendable to prevent extension of the 3′ end of the capture oligonucleotide (e.g., oligo-dT or oligo-dN template) at an internal priming site. Internal priming may result in not capturing the entire length of the poly-A tail in a mRNA or the full length non-polyadenylated RNA. Most 3′ modifications will block extension during PCR, linear amplification or reverse transcription (e.g., a 3′ didexoy nucleotide, spacer, etc.). Non-limiting examples of non-extendable 3′ ends include 3′ddC, 3′ Inverted dT, 3′ C3 spacer, 3′ Amino, and 3′ phosphorylation.

In example embodiments, the capture oligonucleotide can include one or more selectively cleavable bases (e.g., dU nucleotides or riboU nucleotides), such as 1, 2, 3, or 4, preferably, the capture oligonucleotide template includes one selectively cleavable base. As used herein “ribobase” and “ribose base” refer to a nucleotide containing ribose as its pentose component. The most common bases for ribonucleotides are adenine (A), guanine (G), cytosine (C), or uracil (U). As used herein “deoxyU” “dU” refer to a nucleoside that closely resembles the chemical composition of uridine but without the presence of the 2′ hydroxyl group.

### Barcodes

In example embodiments, the capture oligonucleotide includes one or more nucleic acid barcode sequences. In example embodiments, the template switching oligo (TSO) includes one or more nucleic acid barcode sequences. As used herein, the terms “barcode” and “nucleic acid barcode” refer to a short sequence of nucleotides (for example, DNA or RNA) that is used as an identifier for an associated molecule, such as a target molecule and/or target nucleic acid, or as an identifier of the source of an associated molecule, such as a cell-of-origin. A barcode can have a length of at least, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 200, or 300 nucleotides, and can be in single or double-stranded form. Typically, a nucleic acid barcode is used to identify a target molecule and/or target nucleic acid as being from a particular discrete volume, sample, single cell or spatial location, having a particular physical property (for example, affinity, length, sequence, etc.), or having been subject to certain treatment conditions. Thus, a sample barcode is the same for all target nucleic acids in a sample, but different from the sample barcode in any other sample and a cell barcode is the same for all target nucleic acids in a single cell, but different for the cell barcode in any other single cell. In an example embodiment, amplified sequences from single cells or multiple samples can be sequenced together and resolved based on the barcode associated with each cell or sample. Target molecule and/or target nucleic acid can be associated with multiple nucleic acid barcodes to provide information about all of these features (and more). In certain embodiments, barcoding uses an error correcting scheme (T. K. Moon, Error Correction Coding: Mathematical Methods and Algorithms (Wiley, New York, ed. 1, 2005)). In an example embodiment, amplified sequences from single cells can be sequenced together and resolved based on the barcode associated with each cell.

Unique molecular identifiers are a subtype of nucleic acid barcode that can be used, for example, to normalize samples for variable amplification efficiency (See e.g., Islam S. et al., 2014. Nature Methods No: 11, 163-166). The term “unique molecular identifiers” (UMI) as used herein refers to a subtype of nucleic acid barcode used in a method that uses molecular tags to detect and quantify unique amplified products. The UMI sequence is unique to each target nucleic acid in a specific sample. Specific samples may be distinguished by a sample barcode or single cell barcode. A UMI may be used to determine the number of transcripts that gave rise to an amplified product (i.e., counting the number of transcripts). In certain embodiments, the capture oligonucleotide includes a UMI with a random sequence of between 4 and 20 base pairs that is incorporated into the full-length cDNA, which is amplified and sequenced. Each cDNA amplified will have a different random UMI that will indicate that the amplified product originated from that cDNA. Background caused by the fidelity of the amplification process can be eliminated because background representing random error will only be present in single amplification products. In example embodiments, UMI's are designed such that assignment to the original can take place despite up to 4-7 errors during amplification or sequencing.

Barcodes for capture oligonucleotides or TSOs can be generated from a variety of different formats, including bulk synthesized polynucleotide barcodes, randomly synthesized barcode sequences, microarray based barcode synthesis, native nucleotides, partial complement with N-mer, random N-mer, pseudo random N-mer, or combinations thereof. Synthesis of barcodes is described, for example, in U.S. Pat. No. 9,388,465. Barcodes for oligo-dT templates or TSOs can be generated, for example, by split-pool synthesis methods, such as those described, for example, in International Patent Publication Nos. WO 2014/047556 and WO 2014/143158.

### Promoter Sequences

In example embodiments, the capture oligonucleotide or TSO includes a promoter sequence. The promoter sequence is preferably at the 5′ end of the capture oligonucleotide or TSO between the sequence containing one or more barcode sequences and the terminal adapter sequence. The promoter is required to be 5′ of the barcode sequence so that upon transcription from the promoter the barcode sequence is transcribed. The promoter sequence can be used to amplify the full-length cDNA generated by mRNA end to end sequencing (mEE-seq) using in vitro transcription. In vitro transcription is a common route to amplify genetic material and is less prone to certain amplification biases. A number of RNA polymerase promoters may be used for the promoter region of the capture oligonucleotide. Suitable promoter regions will be capable of initiating transcription from an operationally linked DNA sequence in the presence of ribonucleotides and an RNA polymerase under suitable conditions. The promoter region will usually comprise between about 15 and 250 nucleotides, preferably, between about 17 and 60 nucleotides, from a naturally occurring RNA polymerase promoter, a consensus promoter region, or an artificial promoter region, as described in Alberts et al. (1989) in Molecular Biology of the Cell, 2d ed. (Garland Publishing, Inc.). In general, prokaryotic promoters are preferred over eukaryotic promoters, and phage or virus promoters are most preferred. As used herein, the term “operably linked” refers to a functional linkage between the affecting sequence (typically a promoter) and the controlled sequence (the cDNA). The promoter sequence can be from a prokaryotic or eukaryotic source. Representative promoter regions of particular interest include T7, T3 and SP6 as described in Chamberlin and Ryan, The Enzymes (ed. P. Boyer, Academic Press, New York) (1982) pp 87-108. In a preferred embodiment, the RNA polymerase promoter sequence is a T7 RNA polymerase promoter sequence comprising at least nucleotides-17 to +6 of a wild-type T7 RNA polymerase promoter sequence, preferably joined to at least 20, preferably at least 30 nucleotides of upstream flanking sequence, particularly upstream T7 RNA polymerase promoter flanking sequence. Additional downstream flanking sequence, particularly downstream T7 RNA polymerase promoter flanking sequence, e.g., nucleotides +7 to +10, may also be advantageously used. For example, in one particular embodiment, the promoter comprises nucleotides-50 to +10 of a natural class III T7 RNA polymerase promoter sequence.

### Adapter Sequences

Example embodiments include adapters. As used herein, an “adapter” or “adaptor” is a nucleotide sequence added to a target polynucleotide sequence, for example, a polynucleotide sequence comprising primer binding sites for amplification and/or sequencing, and/or functional sequences, such as, a polynucleotide sequence compatible for ligation with a target polynucleotide or a promoter. An adapter may comprise a sequence used for attachment or hybridization to another sequence, such as a barcode sequence. The adapter sequence can include an overhang sequence for hybridization and ligation to a target polynucleotide sequence. The adapter can be a hairpin sequence that includes an overhang sequence for hybridization and ligation to a target polynucleotide sequence.

In example embodiments, adapters are added to both ends of the full-length cDNA generated from the target RNAs, such that the cDNA can be amplified and sequenced. The adapters can be added by including 5′ adapter sequences on the capture oligonucleotide (e.g., oligo-dT or oligo-dN template) and the TSO oligonucleotide (described further herein). Adapters can be added to the full-length cDNA by using a terminal deoxynucleotidyl transferase (TdT), poly(A) polymerase, or poly(U) polymerase to add nucleotides to the 3′ of the first strand synthesis product and using an adapter sequence comprising an overhang complementary to the nucleotides added. A ligase can be used to ligate the adapter to the cDNA. The adapter can be double stranded or a hairpin sequence. Adapters can also be added by template switching mechanisms. Non-limiting example adapters that may be attached to sequences and that allow for amplification and sequencing include the P5 and P7 adapter constructs (such as Illumina) having flow cell binding sites, which allow sequencing library fragments to attach to the flow cell surface, such as in Illumina sequencing.

### Deoxyuracil Glycosylase

In one example embodiment, the systems and methods of the present disclosure include a uracil DNA glycosylase that only has activity on a deoxyuracil present in a DNA: DNA duplex or DNA/RNA heteroduplex. Enzymes in the uracil DNA glycosylase (UDG) superfamily are well known for their role in the removal of deaminated base damage in DNA repair (see, e.g., Lee D H, Liu Y., Lee H W, et al. A structural determinant in the uracil DNA glycosylase superfamily for the removal of uracil from adenine/uracil base pairs. Nucleic Acids Res. 2015;43 (2): 1081-1089; and Xia B., Liu Y., Li W, Brice A R, Dominy B N, Cao W. Specificity and catalytic mechanism in family 5 uracil DNA glycosylase. J Biol Chem. 2014; 289 (26): 18413-18426). In example embodiments, the deoxyuracil glycosylase is a family 5 UDGb. Family 5 UDGb exists in archaca and bacteria, many of which are hyperthermophiles or thermophiles (Xia, et al., 2014). The UDG activity from family 5 UDGb is limited to double-stranded uracil-containing DNA and the activity on A/U base pairs is lower than that on mismatched base pairs (Lec, et al., 2015). Mutations in UDGb can increase its activity toward double-stranded uracil-containing base pairs with the most notable increase occurring on A/U base pairs (Lee, et al., 2015). The AllIN mutation in family 5 UDGb from Thermus thermophiles increases its activity toward double-stranded uracil-containing base pairs with the most notable increase occurring on A/U base pairs (Lee, et al., 2015). In example embodiments, a family 5 UDGb having a mutation in the same position is used. In other example embodiments, any enzyme in the uracil DNA glycosylase (UDG) superfamily that is modified to be limited to activity on double-stranded uracil-containing DNA and not on single stranded templates as described herein can be used.

### Endonuclease

In example embodiments, the systems and methods of the present disclosure include an endonuclease for cleavage of the capture oligonucleotide when it is in an extended double strand DNA molecule. In preferred embodiments, the endonuclease is endonuclease VIII or endonuclease IV. Endonuclease VIII from E. coli acts as both an N-glycosylase and an AP-lyase. Endonuclease IV is an apurinic/apyrimidinic (AP) endonuclease that will hydrolyse intact AP sites in DNA. In an example embodiment, UDG first catalyzes the excision of uracil, leading to the formation of an abasic site. An abasic site is a site in DNA where a base is missing, also known as an apurinic/apyrimidinic (AP) site. This AP-site can then either be cleaved by the lyase activity of specific endonucleases, or chemically. Specific endonucleases with a much higher affinity to abasic sites, include, but are not limited to endonuclease VIII, endonuclease IV, or Exonuclease III. Endonuclease VIII, endonuclease IV, and Exonuclease III have an AP-lyase activity that catalyzes the cleavage of the phosphodiester backbone 3′ and/or 5′ of the AP-site, releasing the base-free deoxyribose, and thus forming a single-nucleotide gap (see, e.g., Hölz K, Pavlic A, Lictard J, Somoza M M. Specificity and Efficiency of the Uracil DNA Glycosylase-Mediated Strand Cleavage Surveyed on Large Sequence Libraries. Sci Rep. 2019; 9 (1): 17822).

### Ribonucleases

In one example embodiment, the systems and methods of the present disclosure include a ribonuclease that selectively cleaves an RNA base in a DNA: DNA duplex, such as RNAscH enzymes. Members of the RNase H family can be found in nearly all organisms, from bacteria to archaea to eukaryotes. In preferred embodiments, the enzyme used is an RNaseH2. In preferred embodiments, the enzyme used is a prokaryote RNaseH2. RNAseH2 selectively cleaves a ribose base in the context of a DNA: DNA duplex leaving a 3′ OH. In prokaryotes, RNase H2 is enzymatically active as a monomeric protein. The heterotrimeric type II ribonuclease H enzyme (RNaseH2) in humans includes the RNase H2 subunit A, RNASEH2B, and RNASEH2C subunits. Both prokaryotic and eukaryotic H2 enzymes can cleave single ribonucleotides in a strand, however, they have slightly different cleavage patterns and substrate preferences: prokaryotic enzymes have lower processivity and hydrolyze successive ribonucleotides more efficiently than ribonucleotides with a 5′ deoxyribonucleotide, while eukaryotic enzymes are more processive and hydrolyze both types of substrate with similar efficiency. The substrate specificity of RNase H2 gives it a role in ribonucleotide excision repair, removing misincorporated ribonucleotides from DNA, in addition to R-loop processing. Any engineered or evolved enzyme capable of similar activity can be used herein.

### Reverse Transcriptase

In example embodiments, reverse transcriptase (RT) is used for RNA-dependent DNA polymerase activity and DNA-dependent DNA polymerase activity. In preferred embodiments, the RT has an associated terminal deoxynucleotidyl transferase (TdT)-like activity, which can add nontemplated nucleotides to the 3′ ends of DNA. In preferred embodiments, the RT adds three nontemplated protruding nucleotides. Non-limiting RT enzymes include Moloney murine leukemia virus (MMLV) and avian myeloblastosis virus (AMV) reverse transcriptases, both commercially available (see, e.g., Chen D, Patton J T. Reverse transcriptase adds nontemplated nucleotides to cDNAs during 5′-RACE and primer extension. Biotechniques. 2001;30 (3): 574-582). Certain reverse transcriptase enzymes (e.g., Avian Myeloblastosis Virus (AMV) Reverse Transcriptase and Moloney Murine Leukemia Virus (M-MuLV, MMLV) Reverse Transcriptase) can synthesize a complementary DNA strand using both RNA (cDNA synthesis) and single-stranded DNA (ssDNA) as a template. Thus, in some embodiments, the reverse transcription reaction can use an enzyme (reverse transcriptase) that is capable of using both RNA and ssDNA as the template for an extension reaction, e.g., an AMV or MMLV reverse transcriptase. “Reverse transcriptase” includes not only naturally occurring enzymes, but all such modified derivatives thereof, including also derivatives of naturally-occurring reverse transcriptase enzymes.

In example embodiments, xenopolymerases with reverse transcriptase activity can be used as the reverse transcriptase. An example xenopolymerase is RTX (see, e.g., Ellefson J W, Gollihar J, Shroff R, Shivram H, Iyer V R, Ellington A D. Synthetic evolutionary origin of a proofreading reverse transcriptase. Science. 2016; 352 (6293): 1590-1593; and Choi W S, He P, Pothukuchy A, Gollihar J, Ellington A D, Yang W. How a B family DNA polymerase has been evolved to copy RNA. Proc Natl Acad Sci USA. 2020; 117 (35): 21274-21280). The evolutionarily distinct reverse transcription xenopolymerase (RTX) actively proofreads on DNA and RNA templates, which greatly improves RT fidelity.

### Template Switching Oligo (TSO)

In example embodiments, a template switching oligonucleotide (TSO) is included in the system. A “template switching oligonucleotide” is an oligonucleotide that hybridizes to untemplated nucleotides added by a reverse transcriptase (e.g., enzyme with terminal transferase activity) during reverse transcription. In some embodiments, a template switching oligonucleotide hybridizes to untemplated poly(C) nucleotides added by a reverse transcriptase. Template switching is the ability of the MMLV reverse transcriptase to introduce a few untemplated nucleotides, predominantly 2-5 cytosines, when it reaches the 5′-end of the RNA template, corresponding to the 3′-end of the newly synthesized cDNA strand (see, e.g., Picelli S., Faridani O R, Bjorklund A K, Winberg G., Sagasser S., Sandberg R. Full-length RNA-seq from single cells using Smart-seq2. Nature protocols 2014; 9:171-81). These extra nucleotides work as a docking site for a helper oligonucleotide (“Template Switching Oligonucleotide”, or TSO) that, in the first Smart-seq kit, carried 3 riboguanosines at its 3′-end. The reverse transcriptase is then able to “switch template” (from mRNA to the DNA of the TSO) and synthesize a complementary DNA strand using the helper oligonucleotide as template. Thus, template switching makes possible the introduction of an arbitrary sequence at the end of the transcript and, along with the known sequence located at the 5′-end of the oligo-dT template, allows the efficient amplification of all the transcripts in a cell using a PCR step.

In one example embodiment, a LNA is used in the TSO. The TSO in the Smart-seq2 method replaces the terminal riboguanosine with a locked nucleic acid (LNA)-modified deoxyguanosine. Locked nucleotides are characterized by an internal bond between the 02′ and the C4′ of the furanose ring, linked by a methylene group. The modification introduces a conformational lock in the molecule, which nonetheless still retains the physical properties of the native nucleic acid. Two interesting properties of LNAs are advantageous for this application: the enhanced thermal stability of the LNA monomers and their ability to anneal strongly to the untemplated 3′ extension of the cDNA.

In one example embodiment, a 3′-deoxyguanosine is used in the TSO. The 3′-deoxyguanosine TSO prevents internal priming/strand invasion.

In example embodiments, the 3′ end of the TSO is NGG (where ‘N’ can be either A or C or T). In example embodiments, the 3′ end of the TSO is GGG. In studies looking at the base composition of non-template nucleotide addition, a clear preference of ribo base guanosine at 3 end of TSO was observed. However, the guanosine preference was reduced with increasing distance from 3 end (see, e.g., Thesis of Saiful Islam, Karolinska Institute, 2013, entitled From Single-Cell Transcriptomics To Single-Molecule Counting).

Template switching oligonucleotides can include deoxyribonucleic acids; ribonucleic acids; modified nucleic acids including 2-aminopurine, 2,6-diaminopurine (2-amino-dA), inverted dT, 5-methyl dC, 2′-deoxyInosine, Super T (5-hydroxybutynl-2′-deoxyuridine), Super G (8-aza-7-deazaguanosine), locked nucleic acids (LNAs), unlocked nucleic acids (UNAs, e.g., UNA-A, UNA-U, UNA-C, UNA-G), Iso-dG, Iso-dC, 2′ fluoro bases (e.g., Fluoro C, Fluoro U, Fluoro A, and Fluoro G), or any combination of the foregoing.

In some embodiments, the length of a template switching oligonucleotide can be at least about 1, 2, 10, 20, 50, 75, 100, 150, 200, or 250 nucleotides or longer. In some embodiments, the length of a template switching oligonucleotide can be at most about 2, 10, 20, 50, 100, 150, 200, or 250 nucleotides or longer.

### Solid Supports

In example embodiments, capture oligonucleotides or TSOs can be attached to a solid support or surface, such as, a bead, a solid array, a slide, or a coverslip. In some examples, capture oligonucleotides or TSOs can be encapsulated within, embedded within, or layered on a surface of a permeable composition (e.g., any of the substrates described herein). For example, capture oligonucleotides or TSOs can be encapsulated or disposed within a permeable bead (e.g., a gel bead) or attached to the surface of a bead. In some examples, capture oligonucleotides or TSOs can be encapsulated within, embedded within, or layered on a surface of a substrate (e.g., any of the exemplary substrates described herein, such as a hydrogel or a porous membrane). For example, in various embodiments, featuring a solid or semisolid support, to which capture oligonucleotides or TSOs are attached, the target molecule receives a nucleic acid barcode that identifies the originating solid or semisolid support or the location on the solid support.

### Beads

In example embodiments, the solid support is a bead (i.e., particle). In example embodiments, beads include any bead used for single cell methods as described further herein. Non-limiting examples of beads include hydrogel particles (polyacrylamide, agarose, etc.), colloidal particles (polystyrene, magnetic or polymer particle, etc.), any bead which can leverage phosphoramidate chemistry such as those used in oligonucleotide synthesis known to those skilled in the art (e.g., methylacrylates, polysterenes, polyacrylamides, polyethylenglycols), paramagnetic beads, and magnetic beads. In example embodiments, the beads are 1 to 500 micrometer in size, or other dimensions such as those described herein.

In example embodiments, the bead may be a hydrogel particle (see, e.g., Int. Pat. Apl. Pub. No. WO 2008/109176 for examples of hydrogel particles, including hydrogel particles containing DNA). Examples of hydrogels include, but are not limited to agarose or acrylamide-based gels, such as polyacrylamide, poly-N-isopropylacrylamide, or poly N-isopropylpolyacrylamide. For example, an aqueous solution of a monomer may be dispersed in a droplet, and then polymerized, e.g., to form a gel.

In example embodiments, the beads may comprise one or more polymers. Exemplary polymers include, but are not limited to, polystyrene (PS), polycaprolactone (PCL), polyisoprene (PIP), poly(lactic acid), polyethylene, polypropylene, polyacrylonitrile, polyimide, polyamide, and/or mixtures and/or co-polymers of these and/or other polymers. In addition, in some cases, the particles may be magnetic, which could allow for the magnetic manipulation of the particles. For example, the particles may comprise iron or other magnetic materials. The particles could also be functionalized so that they could have other molecules attached, such as proteins, nucleic acids or small molecules. In some embodiments, the particle may be fluorescent.

Beads comprising the capture oligonucleotides or TSOs of the present disclosure can be obtained by any previously described method. For example, the capture oligonucleotides or TSOs can be directly synthesized on the beads, such that barcodes can be generated by random synthesis (see, e.g., Macosko et al., 2015, “Highly Parallel Genome-wide Expression Profiling of Individual Cells Using Nanoliter Droplets” Cell 161, 1202-1214; and International patent application number PCT/US2015/049178, published as WO 2016/040476 on Mar. 17, 2016). In example embodiments, beads are obtained by 1) performing reverse phosphoramidite synthesis on the surface of the bead to synthesize the 5′ end of the capture oligonucleotides from a linker on the bead; 2) performing reverse phosphoramidite synthesis on the surface of the bead in a pool-and-split fashion, such that in each cycle of synthesis the beads are split into four reactions with one of the four canonical nucleotides (T, C, G, or A) or unique oligonucleotides; 3) repeating this process a large number of times, at least two, and optimally more than twelve, such that, in the latter, there are more than 16 million unique barcodes on the surface of each bead in the pool; and 4) synthesizing or attaching (e.g., ligating) the 3′ end of the capture oligonucleotides comprising dU, poly-dT or poly-dN and blocked 3′ end. For synthesis, the bead has to be a material that can be maintained during organic synthesis. Non-limiting examples include any bead which can leverage phosphoramidate chemistry such as those used in oligonucleotide synthesis known to those skilled in the art.

In another example, the capture oligonucleotides or TSOs can be synthesized by linking oligonucleotides to beads followed by split-pool hybridization and extension to generate unique cell barcodes for each bead (see, e.g., Klein et al., 2015, “Droplet Barcoding for Single-Cell Transcriptomics Applied to Embryonic Stem Cells” Cell 161, 1187-1201; and International patent application number PCT/US2016/027734, published as WO 2016/168584A1 on Oct. 20, 2016). In example embodiments, a nucleic acid barcode can be constructed in combinatorial fashion by combining randomly selected indices (for example, about 1, 2, 3, 4, 5, 6, 7, 8, 9, or 10 indexes). Each such index is a short sequence of nucleotides (for example, DNA, RNA, or a combination thereof) having a distinct sequence. An index can have a length of about, for example, 4, 5, 6, 7, 8, 9, 10, 11, 12, 13, 14, 15, 16, 17, 18, 19, 20, 21, 22, 23, 24, or 25 bp or nt. Accordingly, in some embodiments, the possible barcodes that are used are formed from one or more separate “pools” of barcode elements that are then joined together to produce the final barcode, e.g., using a split-and-pool approach. A pool may contain, for example, at least about 300, at least about 500, at least about 1,000, at least about 3,000, at least about 5,000, or at least about 10,000 distinguishable barcodes. For example, a first pool may contain x1 elements and a second pool may contain x2 elements; forming a barcode containing an element from the first pool and an element from the second pool may yield, e.g., x1x2 possible barcodes that could be used. It should be noted that x1 and x2 may or may not be equal. This process can be repeated any number of times; for example, the barcode may include elements from a first pool, a second pool, and a third pool (e.g., producing x1X2x3 possible barcodes), or from a first pool, a second pool, a third pool, and a fourth pool, etc. Accordingly, due to the potential number of combinations, even a relatively small number of barcode elements can be used to produce a much larger number of distinguishable barcodes. A UMI can either be added before or after synthesis of the bead identifying barcode (cell barcode) by the split pool method. The UMI may be present on the 5′ end of the capture oligonucleotide or may be present on the last index used for generating the cell barcode.

In another example, the capture oligonucleotides or TSOs can be synthesized by linking the 5′ end of oligonucleotides containing adaptor sequences to beads to generate functionalized beads followed by emulsion PCR using primers containing unique cell barcode sequences (see, e.g., Zheng, et al., 2016, “Haplotyping germline and cancer genomes with high-throughput linked-read sequencing” Nature Biotechnology 34, 303-311; Zheng, et al., 2017, “Massively parallel digital 1 transcriptional profiling of single cells” Nat. Commun. 8, 14049 doi: 10.1038/ncomms14049; International patent publication number WO 2014/210353A2; and Zilionis, et al., 2017, “Single-cell barcoding and sequencing using droplet microfluidics” Nat Protoc. Jan; 12 (1): 44-73). In this embodiment, each emulsion PCR includes a single primer that can hybridize to oligonucleotides on the functionalized beads and comprise a barcode sequence. Thus, after several rounds of amplification the barcode sequence is transferred to every oligonucleotide on the functionalized beads. This results in beads each having a barcode unique to that bead. A UMI sequence, dU sequence and poly-dT or poly-dN sequence can then be added to the beads comprising the cell barcode sequences. In other embodiments, the UMI sequence is included on the functionalized beads before emulsion PCR.

### Slides

In example embodiments, the solid support is a slide or an array on a slide. As used herein the term “slide” includes an “array”, “substrate” or “surface” including a plurality of capture oligonucleotides as described herein. For the spatial array-based analytical methods described herein, a substrate functions as a support for direct or indirect attachment of capture probes (i.e., capture oligonucleotides) to features of the array. In addition, in some embodiments, a substrate (e.g., the same substrate or a different substrate) can be used to provide support to a biological sample, particularly, for example, a thin tissue section. Accordingly, a “substrate” is a support that is insoluble in aqueous liquid and which allows for positioning of biological samples, analytes, features, and/or capture probes on the substrate.

Further, a “substrate” as used herein, and when not preceded by the modifier “chemical”, refers to a member with at least one surface that generally functions to provide physical support for biological samples, analytes, and/or any of the other chemical and/or physical moieties, agents, and structures described herein. Substrates can be formed from a variety of solid materials, gel-based materials, colloidal materials, semi-solid materials (e.g., materials that are at least partially cross-linked), materials that are fully or partially cured, and materials that undergo a phase change or transition to provide physical support. Examples of substrates that can be used in the methods and systems described herein include, but are not limited to, slides (e.g., slides formed from various glasses, slides formed from various polymers), hydrogels, layers and/or films, membranes (e.g., porous membranes), flow cells, cuvettes, wafers, plates, or combinations thereof. In some embodiments, substrates can optionally include functional elements such as recesses, protruding structures, microfluidic elements (e.g., channels, reservoirs, electrodes, valves, seals), and various markings. Slides and arrays for spatial profiling have been described (see, e.g., Visium Spatial Capture Technology, 10× Genomics, Pleasanton, CA; WO 2020/047007A2; WO 2020/123317A2; WO 2020/047005A1; WO 2020/176788A1; and WO 2020/190509A9). The capture probes comprising spatial barcodes can be the capture oligonucleotides comprising spatial barcodes as described herein.

Slides comprising capture oligonucleotides or TSOs can be obtained by synthesizing capture oligonucleotides or TSOs and attaching them to a slide or array. In an example embodiment, specific 5′ oligonucleotide adapters and spatial barcodes are added to specific locations of an array. The rest of the capture oligonucleotide or TSO sequence can then be added to the oligonucleotides to generate the capture oligonucleotides or TSOs with spatial barcodes. In an example embodiment, additional oligonucleotides can be ligated to an in situ synthesized oligonucleotide to generate a capture oligonucleotide or TSO. For example, a primer complementary to a portion of the in situ synthesized oligonucleotide (e.g., a constant sequence in the oligonucleotide) can be used to hybridize an additional oligonucleotide and extend (using the in situ synthesized oligonucleotide as a template e.g., a primer extension reaction) to form a double stranded oligonucleotide and to further create a 3′ overhang. In some embodiments, the 3′ overhang can be created by template-independent ligases (e.g., terminal deoxynucleotidyl transferase (TdT), poly(A) polymerase or poly(U) polymerase). An additional oligonucleotide comprising one or more capture domains can be ligated to the 3′ overhang using a suitable enzyme (e.g., a ligase) and a splint oligonucleotide, to generate a capture oligonucleotide. Thus, in some embodiments, a capture oligonucleotide or TSO is a product of two or more oligonucleotide sequences, (e.g., the in situ synthesized oligonucleotide and the additional oligonucleotide) that are ligated together. In some embodiments, one of the oligonucleotide sequences is an in situ synthesized oligonucleotide.

In some embodiments, gel beads containing oligonucleotides (e.g., barcoded oligonucleotides such as capture probes) can be deposited on a substrate (e.g., a glass slide). In some embodiments, gel pads can be deposited on a substrate (e.g., a glass slide). In some embodiments, gel pads or gel beads are deposited on a substrate in an arrayed format.

Arrays can be prepared by depositing features (e.g., droplets, beads) on a substrate surface to produce a spatially-barcoded array. Methods of depositing (e.g., droplet manipulation) features are known in the art (see, U.S. Patent Application Publication No. 2008/0132429; Rubina, A. Y., et al., Biotechniques. 2003 May; 34 (5): 1008-14, 1016-20, 1022; and Vasiliskov et al. Biotechniques. 1999 Sep.; 27 (3): 592-4, 596-8, 600 passim). A feature can be printed or deposited at a specific location on the substrate (e.g., inkjet printing). In some embodiments, each feature can have a unique oligonucleotide that functions as a spatial barcode. In some embodiments, a feature can be printed or deposited at the specific location using an electric field. A feature can contain a photo-crosslinkable polymer precursor and an oligonucleotide. In some embodiments, the photo-crosslinkable polymer precursor can be deposited into a patterned feature on the substrate (e.g., well). A “photo-crosslinkable polymer precursor” refers to a compound that cross-links and/or polymerizes upon exposure to light. In some embodiments, one or more photoinitiators may also be included to induce and/or promote polymerization and/or cross-linking (see, e.g., Choi et al. Biotechniques. 2019 January; 66 (1): 40-53).

Arrays can be prepared by a variety of methods. In some embodiments, arrays are prepared through the synthesis (e.g., in situ synthesis) of oligonucleotides on the array, or by jet printing or lithography. For example, light-directed synthesis of high-density DNA oligonucleotides can be achieved by photolithography or solid-phase DNA synthesis. To implement photolithographic synthesis, synthetic linkers modified with photochemical protecting groups can be attached to a substrate and the photochemical protecting groups can be modified using a photolithographic mask (applied to specific areas of the substrate) and light, thereby producing an array having localized photo-deprotection. Many of these methods are known in the art, and are described e.g., in Miller et al., “Basic concepts of microarrays and potential applications in clinical microbiology.” Clinical Microbiology Reviews 22.4 (2009): 611-633; US 2013/14111482A; U.S. Pat. No. 9,593,365B2; US 2019/203275; and WO 2018/091676.

### Linkers

In example embodiments, the capture oligonucleotides or TSOs are attached to the solid support as described herein by a linker. In an example embodiment, the linker is capable of being cleaved in the aqueous discrete volume. Thus, cleavage of the linker does not disrupt any of the other reactions in the aqueous volume. In preferred embodiments, the linker is photo-cleavable. Photocleavable linkers are available that can be released by UV irradiation. A PC (Photo-Cleavable) spacer can be placed between DNA bases or between the oligo and a 5′-modifier group. The spacer arm can be cleaved with exposure to UV light in the 300-350 nm spectral range. Cleavage releases the oligo with a 5′-phosphate group. An exemplary photo-cleavable linker is commercially available (Integrated DNA Technologies, Inc., Coralville, Iowa) and shown:

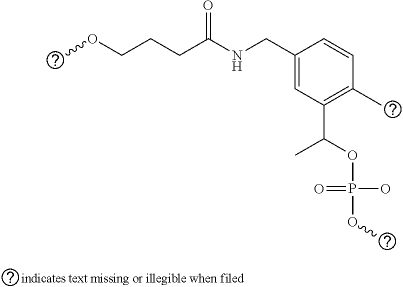

In other example embodiments, the capture oligonucleotides or TSOs may contain one or more cleavable linkers, e.g., that can be cleaved upon application of a suitable stimulus. For example, the cleavable sequence may be a photocleavable linker that can be cleaved by applying light, a chemical cleavable linker that can be cleaved by applying a suitable chemical, or an enzymatically cleavable linker that can be cleaved by applying an enzyme.

Oligonucleotides with photo-sensitive chemical bonds (e.g., photo-cleavable linkers) have various advantages. They can be cleaved efficiently and rapidly (e.g., in nanoseconds and milliseconds). In some cases, photo-masks can be used such that only specific regions of the array are exposed to cleavable stimuli (e.g., exposure to UV light, exposure to light, exposure to heat induced by laser). When a photo-cleavable linker is used, the cleavable reaction is triggered by light, and can be highly selective to the linker and consequently biorthogonal. Non-limiting examples of a photo-sensitive chemical bond that can be used in a cleavage domain include those described in Leriche et al. Bioorg Med Chem. 2012 Jan. 15; 20 (2): 571-82; U.S. Publication No. 2017/0275669; and WO 2020/190509A9.

### Methods

In example embodiments, the systems described herein are used to capture full-length RNA for sequencing. In one example embodiment, full-length RNA sequences are determined for single samples. In this case, the capture oligonucleotides or TSOs only require UMI sequences for identification and/or counting of individual RNAs in the single sample. The reaction can take place in a single tube or reaction vessel. When more than one sample is analyzed, sample barcodes in the capture oligonucleotides or TSOs can be used, such that the capture oligonucleotides or TSOs for different samples include a unique sample barcode. In one example embodiment, full-length RNA sequences are determined for single cells or single nuclei and each single cell or single nuclei is analyzed with capture oligonucleotides or TSOs that include a cell barcode that is unique for the single cell or nuclei.

### Single Cell or Single Nuclei Sequencing

### Plate Based

In example embodiments, single cells or single nuclei are separated into single wells in a plate (see, e.g., Picelli, S. et al., 2014, “Full-length RNA-seq from single cells using Smart-seq2” Nature protocols 9, 171-181, doi: 10.1038/nprot.2014.006). In one embodiment, capture oligonucleotides or TSOs and adapters (e.g., on a TSO or a ligated adapter) can be designed with specific adapter barcode sequences that identify the well the cDNA originated from. In one embodiment, capture oligonucleotides or TSOs can be designed to include barcodes unique to each well in the plate.

### Beads

In one example embodiment, full-length mRNA sequences are determined for single cells or single nuclei and each single cell or single nuclei is analyzed with capture oligonucleotides or TSOs attached to a single bead that includes a cell barcode specific to the bead and that is unique for the single cell or nuclei. In example embodiments, single cells or single nuclei are separated into single droplets or single microwells with single beads.

### Droplets

In example embodiments, single cells or single nuclei are separated into individual droplets comprising single barcoded beads and the one-pot reagents as described herein. Methods of forming droplets comprising single cells or single nuclei and single beads has been described (see, e.g., Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; and Zilionis, et al., 2017, “Single-cell barcoding and sequencing using droplet microfluidics” Nat Protoc. Jan; 12 (1): 44-73).

In example embodiments, single nucleus RNA sequencing is used (see, e.g., Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14 (10): 955-958; International Patent Application No. PCT/US2016/059239, published as WO2017164936 on Sep. 28, 2017; International Patent Application No.PCT/US2018/060860, published as WO/2019/094984 on May 16, 2019; International Patent Application No. PCT/US2019/055894, published as WO/2020/077236 on Apr. 16, 2020; Drokhlyansky, et al., “The enteric nervous system of the human and mouse colon at a single-cell resolution,” bioRxiv 746743; doi: doi.org/10.1101/746743; and Drokhlyansky E, Smillie C S, Van Wittenberghe N, et al. The Human and Mouse Enteric Nervous System at Single-Cell Resolution. Cell. 2020; 182 (6): 1606-1622.c23).

After loading of the beads and cells into droplets, the capture oligonucleotides or TSOs may be released or cleaved from the particles, in accordance with certain aspects. As noted above, any suitable technique may be used to release the oligonucleotides from the droplets, such as light (e.g., if the capture oligonucleotide includes a photocleavable linker), a chemical, or an enzyme, etc. The mRNA can be released from the single cells or nuclei and be captured by the capture oligonucleotides or TSOs. The reagents can then proceed with the one-pot reactions in each individual droplet.

### Microwells

In example embodiments, single cells or single nuclei are separated into individual microwells comprising single barcoded beads and the one-pot reagents as described herein. Methods comprising single cells or single nuclei and single beads in microwells has been described (see, e.g., Gierahn et al., “Seq-Well: portable, low-cost RNA sequencing of single cells at high throughput” Nature Methods 14, 395-398 (2017); and Hughes, et al., “Highly Efficient, Massively-Parallel Single-Cell RNA-Seq Reveals Cellular States and Molecular Features of Human Skin Pathology” bioRxiv 689273; doi: doi.org/10.1101/689273).

### Samples

Single cells or single nuclei can be dissociated from tissues or complex multicellular systems (e.g., organoid, tissue explant, or organ on a chip) (see, e.g., Yin X, Mead B E, Saface H, Langer R, Karp J M, Levy O. Engineering Stem Cell Organoids. Cell Stem Cell. 2016; 18 (1): 25-38; Clevers, Modeling Development and Disease with Organoids, Cell. 2016 Jun. 16;165 (7): 1586-1597; Porter, R. J., Murray, G. I. & McLean, M. H. Current concepts in tumour-derived organoids. Br J Cancer 123, 1209-1218 (2020). doi.org/10.1038/s41416-020-0993-5; Sontheimer-Phelps, A., Hassell, B. A. & Ingber, D. E. Modelling cancer in microfluidic human organs-on-chips. Nat. Rev. Cancer 19, 65-81 (2019); and Wu, Q., Liu, J., Wang, X. et al. Organ-on-a-chip: recent breakthroughs and future prospects. BioMed Eng OnLine 19, 9 (2020); Ingber, D. E. Developmentally inspired human ‘organs on chips’. Development 145, pii: dev156125 (2018); Ghosh S, Prasad M, Kundu K, et al. Tumor Tissue Explant Culture of Patient-Derived Xenograft as Potential Prioritization Tool for Targeted Therapy. Front Oncol. 2019; 9:17; Neil J E, Brown M B, Williams A C. Human skin explant model for the investigation of topical therapeutics. Sci Rep. 2020; 10 (1): 21192; and Grivel J C, Margolis L. Use of human tissue explants to study human infectious agents. Nat Protoc. 2009; 4 (2): 256-269). Tissues or complex multicellular systems include a patient derived organoid (PDO) or patient derived xenograft (PDX). Single cells can be dissociated by any method known in the art, for example enzymatically (e.g., dissociated with TrypLE express (Invitrogen)). Single cells can also be from cultured cells. Single nuclei can also be isolated according to any method known in the art (see, e.g., Drokhlyansky E, Smillie C S, Van Wittenberghe N, et al. The Human and Mouse Enteric Nervous System at Single-Cell Resolution. Cell. 2020; 182 (6): 1606-1622.e23). Both cells and nuclei can be sorted. For example, fluorescence-activated cell sorting (FACS) can be used for plate-based scRNA-seq experiments or for sorting cells or nuclei into tubes for droplet-based scRNA-seq. The systems described herein are compatible with single cells or single nuclei isolated from fresh, formalin-fixed paraffin-embedded, and frozen tissues (see, e.g., WO2020077236A1; and Slyper, M., Porter, C. B. M., Ashenberg, O. et al. (2020). A single-cell and single-nucleus RNA-seq toolbox for fresh and frozen human tumors. Nature Medicine 26 (5): 792-802).

### Spatial Profiling

In example embodiments, spatial profiling of full-length RNA in a tissue sample comprising a plurality of cells is performed. Array-based spatial analysis methods involve the transfer of one or more analytes (e.g., full-length mRNA) from a biological sample to an array of features on a substrate, where each feature is associated with a unique spatial location on the array (e.g., capture oligonucleotides including spatial barcodes). Subsequent analysis of the transferred analytes includes determining the identity of the analytes and the spatial location of each analyte within the biological sample. The spatial location of each analyte within the biological sample is determined based on the spatial barcode to which each mRNA is bound on the array, and the barcode's relative spatial location within the array. One general method is to promote analytes out of a cell and towards the spatially-barcoded array. Another general method is to cleave the spatially-barcoded capture probes from an array, and promote the spatially-barcoded capture probes towards and/or into or onto the biological sample.

In example embodiments, the cells are permeabilized to release mRNA into the aqueous volume of the slide or to allow capture oligonucleotides into the cells, such that the RNA is captured by capture oligonucleotides comprising spatial barcodes that are in proximity to the cells. The cDNAs can be pooled and sequenced. The sequences of the spatial barcodes can be used to deconvolve the location of the RNAs in the tissue sample to generate a three-dimensional map of RNA levels of a tissue sample obtained from a subject, e.g., with a degree of spatial resolution (e.g., single-cell resolution). Methods and compositions for spatial profiling using arrays of spatial barcodes have been described (see, e.g., Visium Spatial Capture Technology, 10× Genomics, Pleasanton, CA; WO 2020/047007A2; WO 2020/123317A2; WO 2020/047005A1; WO 2020/176788A1; and WO 2020/190509A9). The methods can be used for full-length RNAs by using the capture oligonucleotides and systems described herein to obtain spatially resolved full-length RNAs in a single pot reaction as described herein.

In some examples, a cell or a tissue sample including a cell are contacted with capture oligonucleotides attached to a slide (e.g., an array, surface of a substrate), and the cell or tissue sample is permeabilized to allow analytes (e.g., mRNA) to bind to the capture oligonucleotides attached to the substrate. In some embodiments, the plurality of cells is fixed and treated prior to releasing the biological analytes from the cells. In some examples, analytes released from a cell can be actively directed to the capture probes attached to a substrate using a variety of methods, e.g., electrophoresis, chemical gradient, pressure gradient, fluid flow, or magnetic field.

### Samples

Any tissues or complex multicellular systems can be used for full length RNA spatial sequencing (e.g., organoid, tissue explant, or organ on a chip). The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a check swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions.

A sample can be harvested from a subject (e.g., via surgical biopsy, whole subject sectioning), grown in vitro on a growth substrate or culture dish as a population of cells, or prepared as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material (see, e.g., WO 2020/190509A9). In some embodiments, the sample can be prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In some embodiments, a biological sample immobilized on a substrate (e.g., a biological sample prepared using methanol fixation or formalin-fixation and paraffin-embedding (FFPE)) is transferred to a spatial array using a hydrogel. In some embodiments, a hydrogel is formed on top of a biological sample on a substrate (e.g., glass slide). For example, hydrogel formation can occur in a manner sufficient to anchor (e.g., embed) the biological sample to the hydrogel. After hydrogel formation, the biological sample is anchored to (e.g., embedded in) the hydrogel wherein separating the hydrogel from the substrate results in the biological sample separating from the substrate along with the hydrogel. The biological sample can then be contacted with a spatial array, thereby allowing spatial profiling of the biological sample (see, e.g., WO 2020/190509A9).

In some embodiments, a biological sample can be permeabilized to facilitate transfer of analytes out of the sample, and/or to facilitate transfer of species (such as capture oligonucleotides and reagents) into the sample. If a sample is not permeabilized sufficiently, the amount of analyte captured from the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™, Tween-20™, or sodium dodecyl sulfate (SDS)), and enzymes (e.g., trypsin, proteases (e.g., proteinase K). In some embodiments, the detergent is an anionic detergent (e.g., SDS or N-lauroylsarcosine sodium salt solution). In some embodiments, the biological sample can be permeabilized using any of the methods described herein (e.g., using any of the detergents described herein, e.g., SDS and/or N-lauroylsarcosine sodium salt solution) before or after enzymatic treatment (e.g., treatment with any of the enzymes described herein, e.g., trypsin, proteases (e.g., pepsin and/or proteinase K)). Additional methods for sample permeabilization are described, for example, in Jamur et al., Method Mol. Biol. 588:63-66, 2010, the entire contents of which are incorporated herein by reference.

### Kits

In an aspect, kits are provided containing any one or more of the elements discussed herein to allow single-pot End to End mRNA sequencing. For example, a kit may include any embodiment of capture oligonucleotides and TSOs, such as oligo-dT templates for processing mRNA, in a tube or well, a plurality of beads comprising single stranded capture oligonucleotides attached to the beads, or a slide comprising single stranded capture oligonucleotides attached to the slide. Additionally, kits may include a deoxyuracil glyocylase that only has activity on a deoxyuracil present in a DNA: DNA duplex or DNA/RNA heteroduplex (e.g., UDGb, UDGb AllIN), an endonuclease (e.g., endonuclease VIII, endonuclease IV), or a mixture of the two enzymes. Additionally, kits may include an RNaseH2 enzyme. Additionally, kits may include a TSO, adapters, and/or RT. Elements may be provided individually or in combinations, and may be provided in any suitable container, such as a vial, a bottle, or a tube. In some embodiments, the kit includes instructions in one or more languages, for example in more than one language. In some embodiments, a kit comprises one or more reagents for use in a process utilizing one or more of the elements described herein. Reagents may be provided in any suitable container. For example, a kit may provide one or more reaction or storage buffers. Reagents may be provided in a form that is usable in a particular process, or in a form that requires addition of one or more other components before use (e.g., in concentrate or lyophilized form).

Further embodiments are illustrated in the following Examples which are given for illustrative purposes only and are not intended to limit the scope of the invention.

### EXAMPLES

### Example 1-End to End mRNA Sequencing

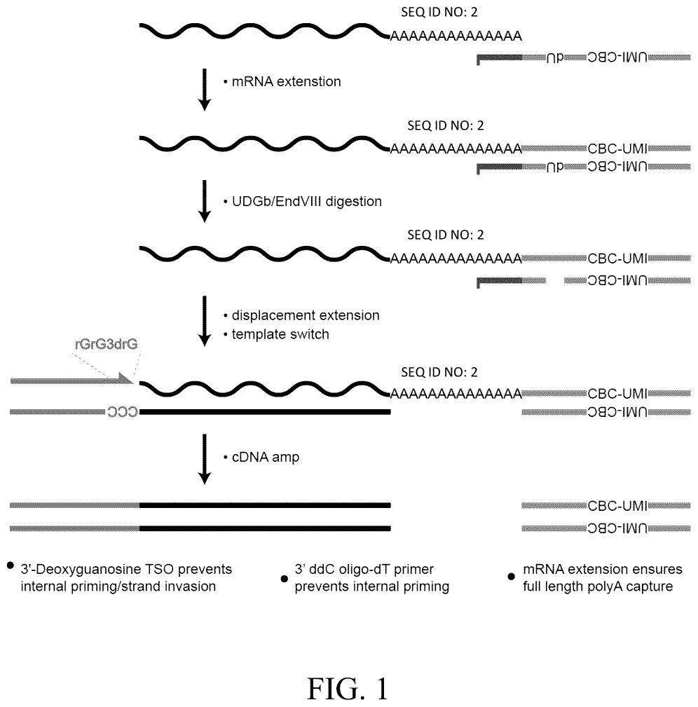

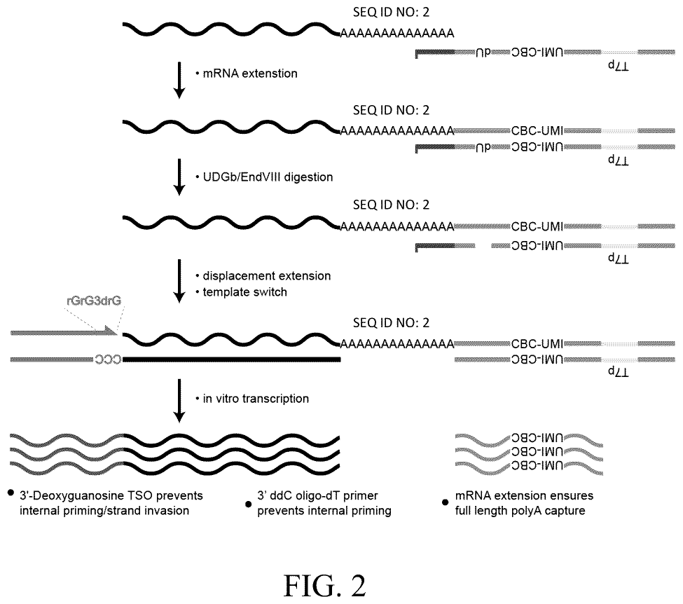

FIG. 1 describes an exemplary embodiment including the main reactions and reaction products that are applicable to any cleavage embodiment described herein. The reactions can all proceed in a single reaction volume or in separate reaction volumes (e.g., droplet, microwell, tube, or surface). The single reaction volume includes the mRNA for capture, the 3′ end blocked oligo-dT template (including the dU sequence and barcodes (UMI and cell barcode), the UDGb and EndVIII enzymes, reverse transcriptase, dNTP's, and a template switching oligonucleotide (TSO). The oligo-dT template is blocked with 3′ ddC to prevent internal priming. The first reaction that occurs is the hybridization of the oligo-dT template to the poly-A tail of the mRNA. The mRNA is used as a primer for extending the mRNA into the oligo-dT template by reverse transcriptase. This generates a double stranded sequence comprising a deoxyuracil. The deoxyuracil glycosylase (UDGb) that is only active on double stranded templates can then excise the dU sequence in the extended double strand sequence to generate an abasic site (a site in DNA where a base is missing, also known as an apurinic/apyrimidinic (AP) site). The endonuclease (EndVIII) cleaves the abasic site resulting in the 3′ end of the oligo-dT template being unblocked. The endonuclease activity produces single-strand breaks on the 5′ side of the apurinic site giving 3′-OH. The oligo-dT template can then be extended by reverse transcriptase using the mRNA as a template. When the reverse transcriptase reaches the 5′ end of the mRNA template switching occurs to introduce an adaptor sequence that can be used for amplification of full-length polyadenylated mRNAs. Thus, full-length polyadenylated mRNAs are captured as cDNA in a single reaction. FIG. 2 describes an exemplary embodiment that includes a T7 promoter in the oligo dT-template for amplification of cDNA using in vitro transcription.

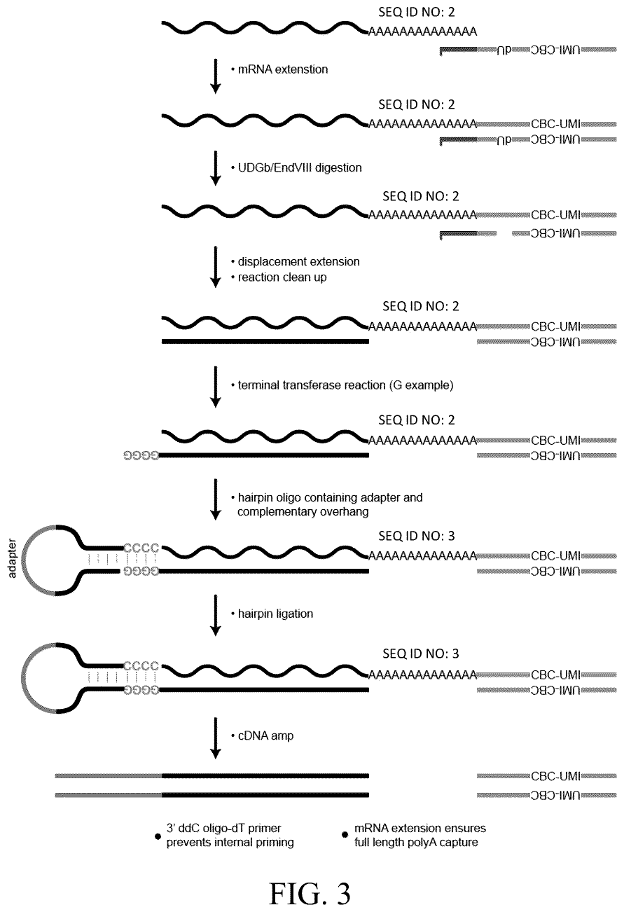

FIG. 3 describes exemplary embodiments that do not require template switching to add an adapter to the 3′ end of the cDNA. The figure details a “tailing” approach used during cDNA synthesis. In short, to add an universal 5′ adapter the following steps are performed: 1) nucleotides are added to the 3′ of the first strand synthesis product using enzymes such as terminal deoxynucleotidyl transferase (TdT), poly(A), or poly(U) polymerase, 2) an oligonucleotide containing both a universal PCR adapter sequence and overhang complementary to the nucleotides added in step 1 are added to the reaction in the presence of a ligase, 3) appropriately hybridized molecules are ligated together and, depending on workflow, undergo either cDNA amplification or in-vitro transcription. The cDNA is generated in single reaction volumes. The first strand cDNA can be pooled before the TdT step because it is barcoded. FIG. 3 shows that the cDNA is 3′ end tailed with Gs and a hairpin adapter is ligated to the cDNA.

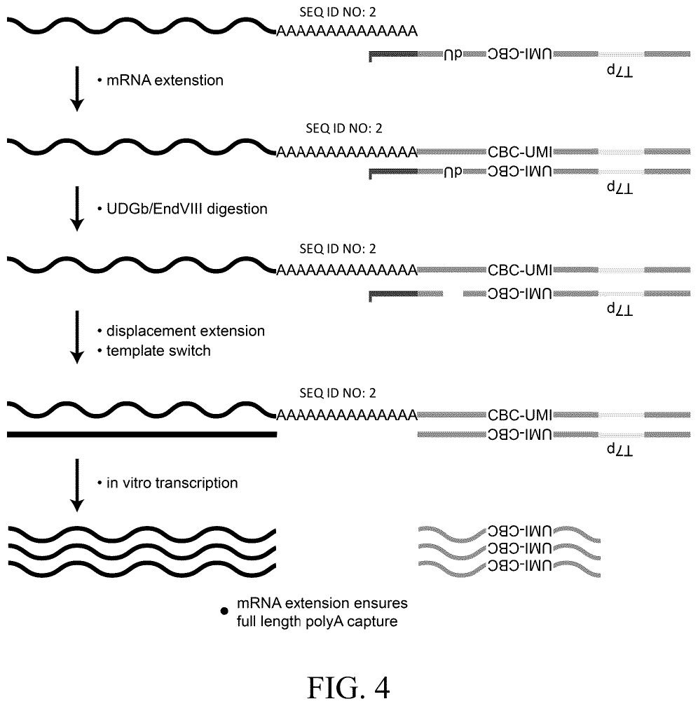

FIG. 4 shows that the cDNA generation does not require template switching or tailing when a T7 promoter is used. FIG. 4 shows an exemplary embodiment that includes a T7 promoter in the oligo dT-template for amplification of cDNA using in vitro transcription. The promoter can be included or not included for the example in FIG. 3.

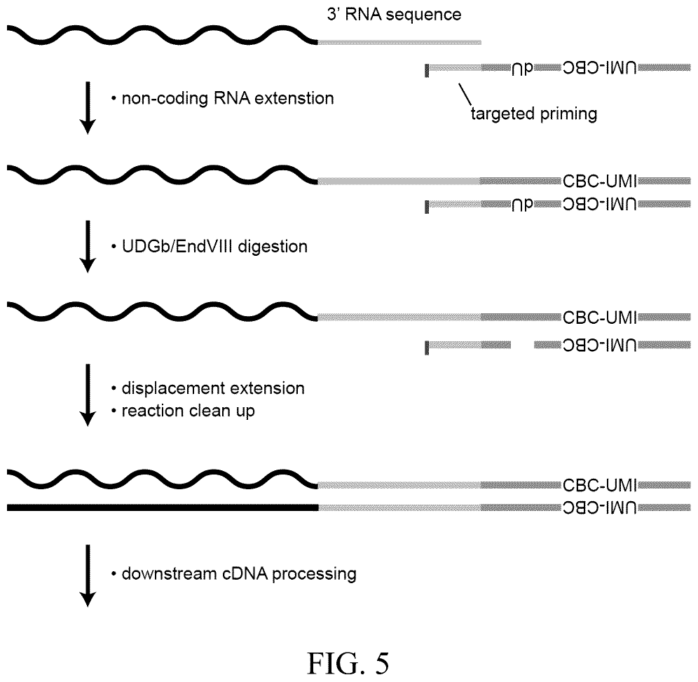

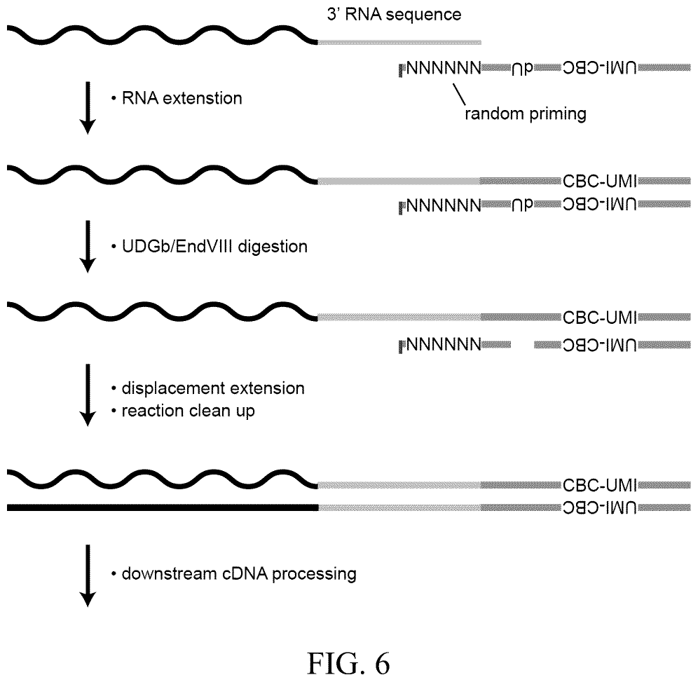

FIG. 5 and FIG. 6 describe exemplary embodiments for using mEE-seq to capture non-polyadenylated RNAs, such as lncRNAs, miRNAs, rRNAs, etc. Instead of an oligo-dT containing primer, the annealing portion is specific for the termini of the non-polyadenylated transcript(s) of interest. For targeting multiple non-polyadenylated transcripts simultaneously, a mix of reverse transcription primers specific for each transcript is used (often referred to as multiplexed capture). Another embodiment is to use a degenerate/random sequence (˜6-20 bp) in place of the oligo-dT portion of the reverse transcriptase primer (capture sequence), enabling capture of transcripts with any potential terminal sequence-inclusive of degraded or non-polyadenylated transcripts. In these embodiments, a promoter can also be included for the examples in FIG. 5 and FIG. 6.

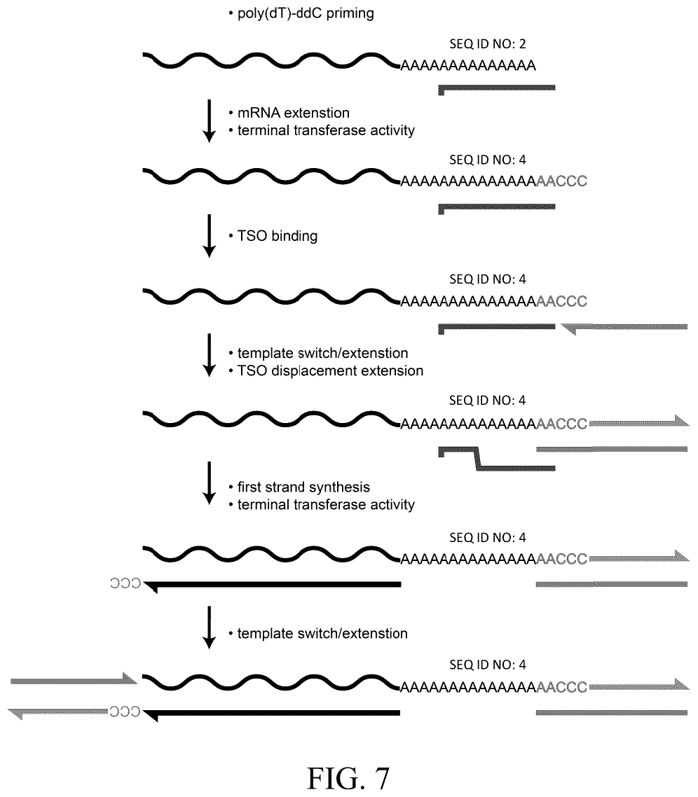

FIG. 7 describes an exemplary embodiment including the main reactions and reaction products that are applicable to any dual TSO embodiment described herein. The reactions can all proceed in a single reaction volume (e.g., droplet, microwell, tube, or surface). Shown is the use of an oligo-dT template containing a 3′ non-extendable end for priming and extension of mRNA on the template oligo by RT, which adds 3 cytosines by terminal transferase activity; template switching using a template switching oligo (TSO) containing 3 guanosine bases, a sequence comprising one or more barcode sequences, and a terminal adapter sequence; extension of the template switch oligo via RT leading to displacement of the oligo-dT template, such that reverse extension can continue until reaching the 5′ of the mRNA, where template switching can occur again. Thus, because the TSO can extend the mRNA after a template switching extension product is generated by extension of the oligo-dT template the reactions can happen in a single reaction volume.

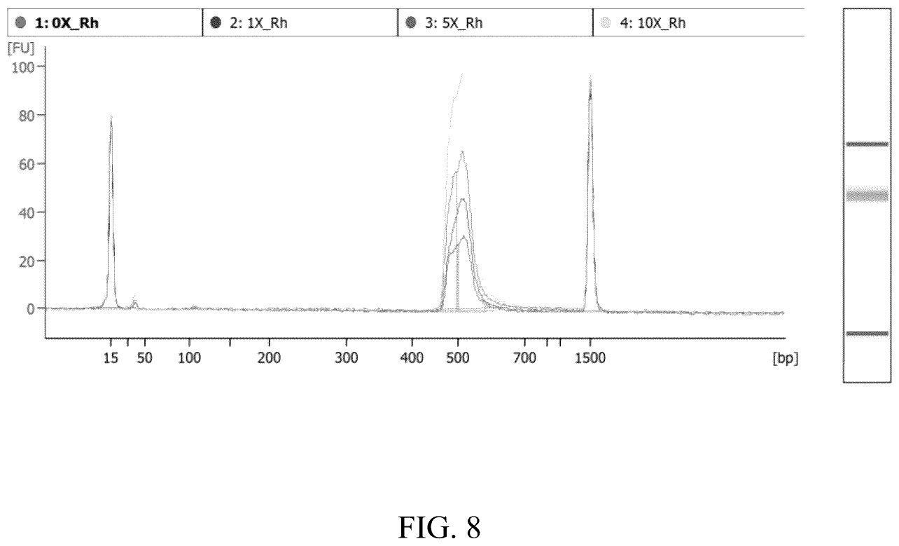

FIG. 8 shows that the addition of RNAscH2 significantly increases the amount of a cDNA product obtained using a 3′ end-blocked oligo-dT template that includes a ribobasc. cDNA synthesis was carried out for 2 hrs at 37C using Maxima H-Reverse Transcriptase in 1× Thermopol buffer using 30 ng of a 452-base polyA-tailed IVT product in the presence of 1 ul 100 uM ‘MEE-Seq’ primer and varying amounts of RNAseH2 enzyme: OX, 1×, 5×, or 10×. To destroy template RNA, all samples were treated with 1 ul RNAse in 1×NEB Buffer 3 for 30 minutes followed by heat inactivation for 15 minutes at 70C. PCR was performed with 1 ul of cDNA product using Deep Vent polymerase for 30 cycles and run neat on a Bioanalyzer DNA1000 chip. Applicants note that RNAscH1 activity intrinsic to MMLV reverse transcriptases results in some cleavage of the RNA base with subsequent cDNA extension and amplification, but the addition of RNAseH2 significantly increased the amount of desired product as expected.

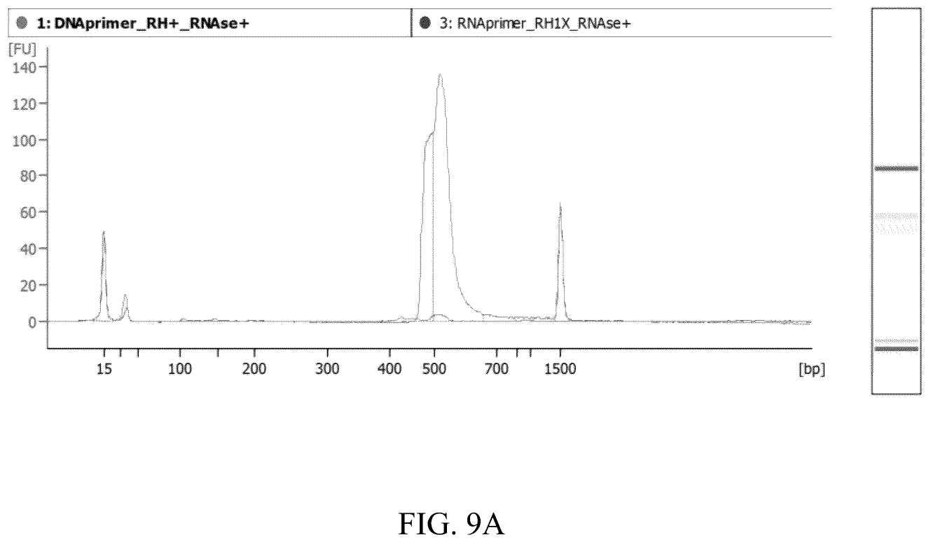

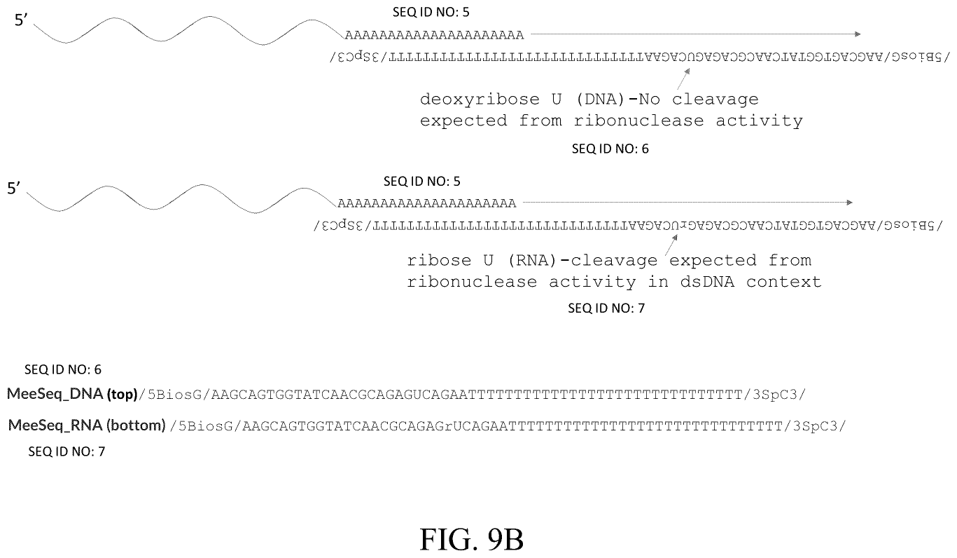

FIG. 9 shows that little to no product is observed when a ribose base (RNA base) is replaced with a deoxy ribose base (DNA base) at the same position using MEE-Seq. cDNA synthesis was carried out for 2 hrs at 37C using Maxima H-Reverse Transcriptase in 1× Thermopol buffer using 300 ng of a 452-base polyA-tailed IVT product in the presence of 1 ul RNAseH2 and 1 ul 100 uM ‘MEE-Seq’ primer’ containing either a ribo-U or deoxy-U. To destroy template RNA, all samples were treated with 1 ul RNAse in 1×NEB Buffer 3 for 30 minutes followed by heat inactivation for 15 minutes at 70C. PCR was performed with 1 ul of cDNA product using Deep Vent polymerase for 30 cycles and run neat on the Bioanalyzer.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1-77. (canceled)

78. A composition comprising: a single stranded capture oligonucleotide comprising from 3′ to 5′:
a non-extendable end,
a capture sequence,
a sequence comprising a selectively cleavable base that can be cleaved in a DNA: DNA duplex or DNA/RNA heteroduplex,
a sequence comprising one or more barcode sequences, and
a terminal adapter sequence.

79. The composition of claim 78, wherein the sequence comprising a selectively cleavable base is a dU sequence.

80. The composition of claim 78, further comprising one or more of:
an enzyme or combination of enzymes capable of cleaving the selectively cleavable base only in a DNA: DNA duplex or DNA/RNA heteroduplex; and/or
a reverse transcriptase.

81. The composition of claim 80, wherein the enzyme or combination of enzymes is a deoxyuracil glycosylase that only has activity on a deoxyuracil present in a DNA: DNA duplex or DNA/RNA heteroduplex and an endonuclease capable of cleavage of an abasic site.

82. The composition of claim 81, wherein the deoxyuracil glycosylase is a family 5 UDGb.

83. The composition of claim 82, wherein the family 5 UDGb comprises an A11IN mutation in the same position as in the family 5 UDGb from Thermus thermophiles.

84. The composition of claim 81, wherein the endonuclease is endonuclease VIII or endonuclease IV.

85. The composition of claim 84, wherein the endonuclease IV is Thermus thermophilus (Tth) endonuclease IV.

86. The composition of claim 78, wherein the sequence comprising a selectively cleavable base is a ribobase comprising sequence.

87. The composition of claim 80, wherein the enzyme or combination of enzymes is RNAseH2.

88. The composition of claim 80, further comprising one or more of:
deoxyribonucleotide triphosphates (dNTPs); and/or
a plurality of RNAs.

89. The composition of claim 88, wherein the capture sequence is an oligo-dT sequence and the plurality of RNAs are a plurality of mRNAs.

90. The composition of claim 88, wherein the capture sequence is an oligo-dN sequence and the plurality of RNAs are a plurality of non-polyadenylated RNAs.

91. The composition of claim 88, wherein the capture sequence is an oligo-dN sequence specific for a non-polyadenylated RNA, a lncRNA, a miRNA, or a rRNA, or is a degenerate/random oligo-dN sequence.

92. The composition of claim 78, wherein:
the composition is comprised in an aqueous discrete volume;
the composition is comprised in more than one aqueous discrete volume, wherein a first aqueous discrete volume comprises at least the single stranded capture oligonucleotide and a plurality of RNAs; or
the composition is comprised in more than one aqueous discrete volume, wherein a first aqueous discrete volume comprises at least the single stranded capture oligonucleotide, deoxyribonucleotide triphosphates (dNTPs), a reverse transcriptase, and a plurality of RNAs, and subsequent aqueous discrete volumes comprise one or more of an enzyme or combination of enzymes capable of cleaving the selectively cleavable base only in a DNA: DNA duplex or DNA/RNA heteroduplex, deoxyribonucleotide triphosphates (dNTPs), and a reverse transcriptase, and any intermediate reaction product,
wherein the aqueous discrete volume or first aqueous discrete volume comprises a plurality of capture oligonucleotides, wherein the one or more barcode sequences for each capture oligonucleotide is a Unique Molecular Identifier (UMI) that is different for each capture oligonucleotide in the plurality of capture oligonucleotides.

93. The composition of claim 92, wherein:
the aqueous discrete volume is a microwell or a droplet;
the capture oligonucleotide or plurality of capture oligonucleotides is attached to a solid support through a linker attached at the 5′ end of the capture oligonucleotides;
the capture oligonucleotide or plurality of capture oligonucleotides is attached to a bead through a linker attached at the 5′ end of the capture oligonucleotides, wherein each aqueous discrete volume comprises no more than one bead; and/or
the capture oligonucleotide or plurality of capture oligonucleotides is attached to a slide through a linker attached at the 5′ end of the capture oligonucleotides and each capture oligonucleotide comprises a spatial barcode that identifies the location of the capture oligonucleotide on the slide.

94. The composition of claim 78, wherein:
the composition further comprises a template switching oligo (TSO) comprising an adapter sequence, a locked nucleic acid (LNA), and/or a 3′-deoxyguanosine.

95. A system for capturing full-length RNAs as cDNA, wherein the system comprises:
a composition comprising a single stranded capture oligonucleotide comprising from 3′ to 5′:
a non-extendable end,
a capture sequence,
a sequence comprising a selectively cleavable base that can be cleaved in a DNA: DNA duplex or DNA/RNA heteroduplex,
a sequence comprising one or more barcode sequences, and
a terminal adapter sequence;
wherein the composition is comprised in one or more aqueous discrete volumes, and wherein the one or more barcode sequences for each capture oligonucleotide further comprises a cell barcode that is the same among capture oligonucleotides in an aqueous discrete volume, but is different among capture oligonucleotides in any other aqueous discrete volume.

96. A method of capturing full-length mRNAs as cDNA, the method comprising:
incubating a composition comprising:
i) a single stranded capture oligonucleotide comprising from 3′ to 5′: a non-extendable end, a capture sequence, a sequence comprising a selectively cleavable base that can be cleaved in a DNA: DNA duplex or DNA/RNA heteroduplex, a sequence comprising one or more barcode sequences, and a terminal adapter sequence;
ii) an enzyme or combination of enzymes capable of cleaving the selectively cleavable base only in a DNA: DNA duplex or DNA/RNA heteroduplex;
iii) deoxyribonucleotide triphosphates (dNTPs);
iv) a reverse transcriptase; and
v) a plurality of RNAs
at one or more temperatures such that mRNA is extended into the capture oligonucleotide by reverse transcriptase, the selectively cleavable base is cleaved in the extended double strand sequence, and the cleaved capture oligonucleotide is extended by reverse transcriptase using the RNA as a template,
wherein the method takes place in a single aqueous discrete volume; or
wherein the method takes place in more than one aqueous discrete volume with or without intervening purification,
whereby full-length RNAs are captured as cDNA in a single reaction or multiple independent reactions.

97. The method of claim 96, further comprising:
a) contacting the cDNA with a terminal deoxynucleotidyl transferase (TdT), a poly(A) polymerase, or a poly(U) polymerase to add nucleotides to the 3′ end of the cDNA to obtain tailed cDNA; and
b) contacting the tailed cDNA with an adapter sequence comprising an overhang complementary to the nucleotides added in (a) and a ligase,
whereby full-length RNAs are captured as cDNA comprising adapters at both ends.
